(19) United States
(12) Patent Application Publication
(10) Pub. No.: US 2016/0032330 A1
(43) Pub. Date: Feb. 4, 2016

(54) P-450-CATALYZED ENANTIOSELECTIVE CYCLOPROPANATION OF ELECTRON-DEFICIENT OLEFINS

(71) Applicant: CALIFORNIA INSTITUTE OF TECHNOLOGY, Pasadena, CA (US)

(72) Inventors: Hans Renata, Arcadia, CA (US); Z. Jane Wang, Los Angeles, CA (US)

(73) Assignee: CALIFORNIA INSTITUTE OF TECHNOLOGY, Pasadena, CA (US)

(21) Appl. No.: 14/732,364

(22) Filed: Jun. 5, 2015

Related U.S. Application Data
(60) Provisional application No. 62/008,285, filed on Jun. 5, 2014.

(51) Int. Cl. C12P 7/62; C12N 9/02

(52) U.S. Cl. CPC C12P 7/62; C12N 9/0071; C12Y 114/14001

(57) ABSTRACT

The present invention pertains to the use of engineered variants of enzyme CYP102A, also known as P450-BM3, for cyclopropanation of olefins containing electron-withdrawing groups. One exemplary enzyme variant, referred to as BM3-HStar, contains five mutations away from wild-type P450-BM3, and demonstrates high activity towards cyclopropanation of olefinic substrates using ethyldiazoacetate (EDA) and other carbene transfer reagents. Products of these reactions are potential precursors of levomilnacipran derivatives, a class of compounds that have been shown to be selective inhibitors of monoamine transporters. In addition, cyclopropanation reactions with the P450-BM3 enzyme variants of the invention can be conducted in whole cells expressing the enzyme variants and can proceed under aerobic conditions.

### CROSS-REFERENCES TO RELATED APPLICATIONS

The present application claims priority to U.S. Provisional Application No. 62/008,285, filed on Jun. 5, 2014, the disclosure of which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant No. EB015846 awarded by the National Institutes of Health. The government has certain rights in the invention.

### BACKGROUND OF THE INVENTION

Expanding the range of synthetic transformations catalyzed by biocatalysts will increase their usage by the synthetic community and aid in the discovery of new biologically active molecules. Enantioselective cyclopropanation is a highly sought after transformation as it can be used to construct multiple stereocenters in one step and synthesize key components of biologically relevant targets. While many catalysts for cyclopropanation using transition metals have been developed, the cost and difficulty of these processes have limited their use on scale in industry.

As an alternative to these methods, a biocatalytic method for cyclopropanation of styrenes in the presence of diazo compounds using engineered variants of cytochrome P450 from Bacillus megaterium (P450-BM3) has been developed (Coelho, et al., Science, 2013, 339, 307). The reaction takes place in water at ambient temperature and the catalyst can carry out tens of thousands of catalytic turnovers. Additionally, mutation of the proximal cysteine ligand in P450-BM3 to serine (C400S) was found to lead to an increase in the Fe^{III}-Fe^{II} redox potential by 140 mV, thereby allowing reduction of the Fe^{III} resting state to the catalytically-active Fe^{II} species under physiological conditions (Coelho, et al., Nat. Chem. Bio., 2013, 9, 485). This key finding allowed for the use of whole cells expressing the enzyme to conduct the reactions, an important advantage of this system because it does not need exogenous reductant or purified protein.

While there have only been a few reported examples of cyclopropanation of electron-deficient olefins with transition metal catalysts, these published reports showed broad generality on a variety of olefins and tolerance to electron-neutral substituents of varying sizes (Wang, et al., Chem. Sci., 2013, 4, 2844; Chen, et al., Tetrahedron Lett., 2008, 49, 6781). In contrast, enzymes often require strong substrate binding for catalysis and thus are highly specific for a particular molecule. For instance, P450-BM3 only undergoes the requisite spin shift for molecular oxygen activation in the presence of a strongly bound substrate like palmitic acid. (McIntosh, et al., Curr. Opin. Chem. Biol., 2014, 19, 126). While this exquisite selectivity can be advantageous in some cases, it is also a significant synthetic limitation because each evolved enzyme can only be used for sterically similar substrates. Accordingly, general enzyme-based biocatalytic methods with broad applicability to a variety of chemical substrates are still needed in the art. The present invention meets this and other needs.

### BRIEF SUMMARY OF THE INVENTION

In a first aspect, the invention provides a reaction mixture for producing a cyclopropanation product. The reaction mixture contains an olefinic substrate, a carbene precursor, and a cytochrome P450 BM3 enzyme variant, wherein the cytochrome P450 BM3 enzyme variant includes a C400H mutation and one or more mutations selected from V78M, L181V, and L437M relative to the amino acid sequence set forth in SEQ ID NO:1.

In a second aspect, the invention provides a method for producing a cyclopropanation product. The method includes forming a reaction mixture containing an olefinic substrate, a carbene precursor, and a cytochrome P450 BM3 enzyme variant under conditions sufficient to produce the cyclopropanation product. The cytochrome P450 BM3 enzyme variant includes a C400H mutation and one or more mutations selected from V78M, L181V, and L437M relative to the amino acid sequence set forth in SEQ ID NO:1.

In a third aspect, the invention provides a cytochrome P450 BM3 enzyme variant including a C400H mutation and one or more mutations selected from V78M, L181V, and L437M relative to the amino acid sequence set forth in SEQ ID NO:1.

Other objects, features, and advantages of the invention will be apparent to one of skill in the art from the following detailed description and figures.

### BRIEF DESCRIPTION OF THE DRAWINGS

FIG. 1 shows the evolution of P450-BM3 for cyclopropanation activity by mutation of the axial ligand at position 400.

FIG. 2 shows a reaction scheme for cyclopropanation reactions conducted according to the methods of the invention.

FIG. 3A shows a set of acrylamide compounds that can be used as substrates for cyclopropanation reactions with BM3-HStar.

FIG. 3B shows a set of acrylate compounds that can be used as substrates for cyclopropanation reactions with BM3-HStar.

FIG. 3C shows the general structure of some substrates for cyclopropanation reactions with BM3-HStar.

### DETAILED DESCRIPTION OF THE INVENTION

### I. Introduction

The present invention is based on the surprising discovery that a mutation at position 400 of cytochrome P450 BM3, the axial ligand of the enzyme's heme moiety, from cysteine to histidine leads to a dramatic increase in the rate of cyclopropanation of olefins (Wang, Z. J., Renata, H., et al. (2014), Angew. Chem. Int. Ed., 53: 6810-6813). See, FIG. 1. Through iterative site-saturation mutagenesis, a P450-BM3 variant referred to herein as BM3-HStar (T268A-C400H-L437W-V78M-L181V) was engineered with five mutations away from wild type P450-BM3. BM3-HStar can catalyze cyclopropanation of acrylamide 1 in greater than 92% yield with 92% enantioselectivity and 2:98 diastereoselectivity (Scheme 1). Conversion of cyclopropane 2 to alcohol 3 constituted a formal synthesis of levomilnacipran, the psychoactive enantiomer of milnacipran and a selective serotonin and norepinephrine reuptake inhibitor recently approved by the US Food and Drug Administration (Shuto, et al., J. Med. Chem., 1995, 38, 2964; Asnis, et al., J. Clin. Psychiatry, 2013, 74, 242). The BM3-Hstar variant has also been found to be a more general catalyst for a broad variety of acrylate and acrylamide substrates. Since previous SAR studies of milnacipran analogs have shown that many molecules within this family are active against monoamine transporters (Tamiya, et al., Bioorg. Med. Chem. Lett., 2008, 18, 3328; Bonnaud, et al., J. Med. Chem., 1987, 30, 818), BM3-Hstar can be employed as a biocatalyst for enantioselective synthesis of levomilnacipran analogs and other useful compounds.

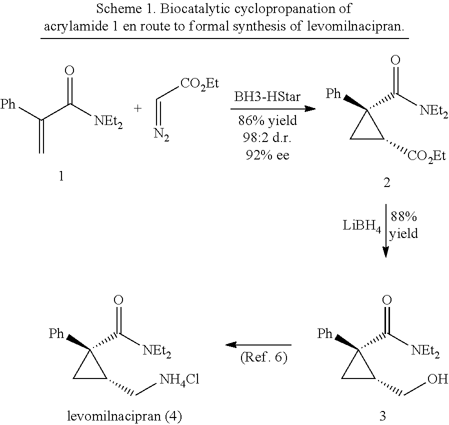

### II. Definitions

The following definitions and abbreviations are to be used for the interpretation of the invention. The term “invention” or “present invention” as used herein is a non-limiting term and is not intended to refer to any single embodiment but encompasses all possible embodiments.

As used herein, the terms “comprises,” “comprising,” “includes,” “including,” “has,” “having, “contains,” “containing,” or any other variation thereof, are intended to cover a non-exclusive inclusion. A composition, mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive “or” and not to an exclusive “or.”

“About” and “around,” as used herein to modify a numerical value, indicate a defined range around that value. If “X” were the value, “about X” or “around X” would generally indicate a value from 0.95X to 1.05X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.98X.” When the quantity “X” only includes whole-integer values (e.g., “X carbons”), “about X” or “around X” indicates from (X−1) to (X+1). In such cases, “about X” or “around X” specifically indicates at least the values X, X−1, and X+1.

The term “cyclopropanation (enzyme) catalyst” or “enzyme with cyclopropanation activity” refers to any and all chemical processes catalyzed by enzymes, by which substrates containing at least one carbon-carbon double bond can be converted into cyclopropane products by using diazo reagents as carbene precursors.

The terms “engineered heme enzyme” and “heme enzyme variant” include any heme-containing enzyme comprising at least one amino acid mutation with respect to wild-type and also include any chimeric protein comprising recombined sequences or blocks of amino acids from two, three, or more different heme-containing enzymes.

The terms “engineered cytochrome P450” and “cytochrome P450 variant” include any cytochrome P450 enzyme comprising at least one amino acid mutation with respect to wild-type and also include any chimeric protein comprising recombined sequences or blocks of amino acids from two, three, or more different cytochrome P450 enzymes.

The term “whole cell catalyst” includes microbial cells expressing heme-containing enzymes, wherein the whole cell catalyst displays cyclopropanation activity.

As used herein, the terms “porphyrin” and “metal-substituted porphyrins” include any porphyrin that can be bound by a heme enzyme or variant thereof. In particular embodiments, these porphyrins may contain metals including, but not limited to, Fe, Mn, Co, Cu, Rh, and Ru.

The terms “carbene equivalent” and “carbene precursor” include molecules that can be decomposed in the presence of metal (or enzyme) catalysts to structures that contain at least one divalent carbon with only 6 valence shell electrons and that can be transferred to C═C bonds to form cyclopropanes or to C—H or heteroatom-H bonds to form various carbon ligated products.

The terms “carbene transfer” and “formal carbene transfer” as used herein include any chemical transformation where carbene equivalents are added to C═C bonds, carbon-heteroatom double bonds or inserted into C—H or heteroatom-H substrates.

As used herein, the terms “microbial,” “microbial organism” and “microorganism” include any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. Also included are cell cultures of any species that can be cultured for the production of a chemical.

As used herein, the term “non-naturally occurring”, when used in reference to a microbial organism or enzyme activity of the invention, is intended to mean that the microbial organism or enzyme has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary non-naturally occurring microbial organism or enzyme activity includes the cyclopropanation activity described above.

As used herein, the term “anaerobic”, when used in reference to a reaction, culture or growth condition, is intended to mean that the concentration of oxygen is less than about 25 μM, preferably less than about 5 μM, and even more preferably less than 1 μM. The term is also intended to include sealed chambers of liquid or solid medium maintained with an atmosphere of less than about 1% oxygen. Preferably, anaerobic conditions are achieved by sparging a reaction mixture with an inert gas such as nitrogen or argon.

As used herein, the term “exogenous” is intended to mean that the referenced molecule or the referenced activity is introduced into the host microbial organism. The term as it is used in reference to expression of an encoding nucleic acid refers to the introduction of the encoding nucleic acid in an expressible form into the microbial organism. When used in reference to a biosynthetic activity, the term refers to an activity that is introduced into the host reference organism.

The term “heterologous” as used herein with reference to molecules, and in particular enzymes and polynucleotides, indicates molecules that are expressed in an organism other than the organism from which they originated or are found in nature, independently of the level of expression that can be lower, equal or higher than the level of expression of the molecule in the native microorganism.

On the other hand, the term “native” or “endogenous” as used herein with reference to molecules, and in particular enzymes and polynucleotides, indicates molecules that are expressed in the organism in which they originated or are found in nature, independently of the level of expression that can be lower equal or higher than the level of expression of the molecule in the native microorganism. It is understood that expression of native enzymes or polynucleotides may be modified in recombinant microorganisms.

The term “homolog,” as used herein with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Homologs most often have functional, structural, or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the amino acid sequence encoded by a gene has a similar amino acid sequence to that of the second gene. Alternatively, a protein has homology to a second protein if the two proteins have “similar” amino acid sequences. Thus, the term “homologous proteins” is intended to mean that the two proteins have similar amino acid sequences. In particular embodiments, the homology between two proteins is indicative of its shared ancestry, related by evolution.

The terms “analog” and “analogous” include nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

As used herein, the term “electron withdrawing group” refers to an atom or substituent that has an ability to acquire electron density from an olefin or other atoms or substituents. An “electron withdrawing group” is capable of withdrawing electron density relative to that of hydrogen if the hydrogen atom occupied the same position on the molecule. The term “electron withdrawing group” is well understood by those of skill in the art and is discussed, for example, in Advanced Organic Chemistry by J. March, John Wiley & Sons, New York, N.Y., (1985). Examples of electron withdrawing groups include, but are not limited to, halo (e.g., fluorine, chlorine, bromine, iodine), nitro, carboxy, amido, acyl, cyano, aryl, heteroaryl, —OC(A)₃, —C(A)₃, —C(A)₂-O—C(A′)₃, —(CO)-Q, —SO₂—C(A)₃, —SO₂-aryl, —C(NQ)Q, —CH═C(Q)₂, and —C≡C-Q; in which each A and A′ is independently H, halo, —CN, —NO₂, —OH, or C₁₋₄ alkyl optionally substituted with 1-3 halo, —OH, or NO₂; and Q is selected from H, —OH, and alkyl optionally substituted with 1-3 halo, —OH, —O-alkyl, or —O-cycloalkyl.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can refer to alkyl groups having up to 20 carbons atoms, such as, but not limited to heptyl, octyl, nonyl, decyl, etc. Alkyl groups can be optionally substituted with one or more moieties selected from halo, hydroxy, amino, alkylamino, alkoxy, haloalkyl, carboxy, amido, nitro, oxo, and cyano.

As used herein, the term “alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be optionally substituted with one or more moieties selected from halo, hydroxy, amino, alkylamino, alkoxy, haloalkyl, carboxy, amido, nitro, oxo, and cyano.

As used herein, the term “alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be optionally substituted with one or more moieties selected from halo, hydroxy, amino, alkylamino, alkoxy, haloalkyl, carboxy, amido, nitro, oxo, and cyano.

As used herein, the term “aryl” refers to an aromatic carbon ring system having any suitable number of ring atoms and any suitable number of rings. Aryl groups can include any suitable number of carbon ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be optionally substituted with one or more moieties selected from halo, hydroxy, amino, alkylamino, alkoxy, haloalkyl, carboxy, amido, nitro, oxo, and cyano.

As used herein, the term “cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, and C₆₋₈. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. Cycloalkyl groups can be optionally substituted with one or more moieties selected from halo, hydroxy, amino, alkylamino, alkoxy, haloalkyl, carboxy, amido, nitro, oxo, and cyano.

As used herein, the term “heterocyclyl” refers to a saturated ring system having from 3 to 12 ring members and from 1 to 4 heteroatoms selected from N, O and S. Additional heteroatoms including, but not limited to, B, Al, Si and P can also be present in a heterocycloalkyl group. The heteroatoms can be oxidized to form moieties such as, but not limited to, —S(O)— and —S(O)₂—. Heterocyclyl groups can include any number of ring atoms, such as, 3 to 6, 4 to 6, 5 to 6, 4 to 6, or 4 to 7 ring members. Any suitable number of heteroatoms can be included in the heterocyclyl groups, such as 1, 2, 3, or 4, or 1 to 2, 1 to 3, 1 to 4, 2 to 3, 2 to 4, or 3 to 4. Examples of heterocyclyl groups include, but are not limited to, aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. Heterocyclyl groups can be optionally substituted with one or more moieties selected from halo, hydroxy, amino, alkylamino, alkoxy, haloalkyl, carboxy, amido, nitro, oxo, and cyano.

As used herein, the term “heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms including, but not limited to, B, Al, Si and P can also be present in a heteroaryl group. The heteroatoms can be oxidized to form moieties such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. Examples of heteroaryl groups include, but are not limited to, pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. Heteroaryl groups can be optionally substituted with one or more moieties selected from halo, hydroxy, amino, alkylamino, alkoxy, haloalkyl, carboxy, amido, nitro, oxo, and cyano.

As used herein, the term “alkoxy” refers to an alkyl group having an oxygen atom that connects the alkyl group to the point of attachment: i.e., alkyl-O—. As for alkyl group, alkoxy groups can have any suitable number of carbon atoms, such as C₁₋₆ or C₁₋₄. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. Alkoxy groups can be optionally substituted with one or more moieties selected from halo, hydroxy, amino, alkylamino, alkoxy, haloalkyl, carboxy, amido, nitro, oxo, and cyano.

As used herein, the term “alkylthio” refers to an alkyl group having a sulfur atom that connects the alkyl group to the point of attachment: i.e., alkyl-S—. As for alkyl groups, alkylthio groups can have any suitable number of carbon atoms, such as C₁₋₆ or C₁₋₄. Alkylthio groups include, for example, methoxy, ethoxy, propoxy, iso-propoxy, butoxy, 2-butoxy, iso-butoxy, sec-butoxy, tert-butoxy, pentoxy, hexoxy, etc. Alkylthio groups can be optionally substituted with one or more moieties selected from halo, hydroxy, amino, alkylamino, alkoxy, haloalkyl, carboxy, amido, nitro, oxo, and cyano.

As used herein, the terms “halo” and “halogen” refer to fluorine, chlorine, bromine and iodine.

As used herein, the term “haloalkyl” refers to an alkyl moiety as defined above substituted with at least one halogen atom.

As used herein, the term “alkylsilyl” refers to a moiety —SiR₃, wherein at least one R group is alkyl and the other R groups are H or alkyl. The alkyl groups can be substituted with one more halogen atoms.

As used herein, the term “acyl” refers to a moiety —C(O)R, wherein R is an alkyl group.

As used herein, the term “oxo” refers to an oxygen atom that is double-bonded to a compound (i.e., 0=).

As used herein, the term “carboxy” refers to a moiety —C(O)OH. The carboxy moiety can be ionized to form the carboxylate anion.

As used herein, the term “amino” refers to a moiety —NR₃, wherein each R group is H or alkyl.

As used herein, the term “amido” refers to a moiety —NRC(O)R or —C(O)NR₂, wherein each R group is H or alkyl.

### III. Description of the Embodiments

In a first aspect, the invention provides a reaction mixture for producing a cyclopropanation product. The reaction mixture contains an olefinic substrate, a carbene precursor, and a cytochrome P450 BM3 enzyme variant, wherein the cytochrome P450 BM3 enzyme variant includes a C400H mutation and one or more mutations selected from V78M, L181V, and L437M relative to the amino acid sequence set forth in SEQ ID NO:1.

A. Cytochrome P450 Enzyme Variants

The cytochrome P450 BM3 enzyme variants used in the methods of the invention belong to the cytochrome P450 family, a large superfamily of heme-thiolate proteins involved in the metabolism of a wide variety of both exogenous and endogenous compounds. Cytochrome P450 enzymes usually act as the terminal oxidase in multicomponent electron transfer chains, such as P450-containing monooxygenase systems. Members of the cytochrome P450 enzyme family catalyze myriad oxidative transformations, including, e.g., hydroxylation, epoxidation, oxidative ring coupling, heteratom release, and heteroatom oxygenation (E. M. Isin et al., Biochim. Biophys. Acta 1770, 314 (2007)). The active site of these enzymes contains an Fe^{III}-protoporphyrin IX cofactor (heme) ligated proximally by a conserved cysteine thiolate (M. T. Green, Current Opinion in Chemical Biology 13, 84 (2009)). The remaining axial iron coordination site is occupied by a water molecule in the resting enzyme, but during native catalysis, this site is capable of binding molecular oxygen. In the presence of an electron source, typically provided by NADH or NADPH from an adjacent fused reductase domain or an accessory cytochrome P450 reductase enzyme, the heme center of cytochrome P450 activates molecular oxygen, generating a high valent iron(IV)-oxo porphyrin cation radical species intermediate and a molecule of water.

The bacterial cytochrome P450 BM3 from Bacillus megaterium is a water soluble, long-chain fatty acid monooxygenase. The native P450 BM3 protein is comprised of a single polypeptide chain of 1048 amino acids and can be divided into 2 functional subdomains (see, L. O. Narhi et al., J. Biol. Chem. 261, 7160 (1986)). An N-terminal domain, amino acid residues 1-472, contains the heme-bound active site and is the location for monoxygenation catalysis. The remaining C-terminal amino acids encompass a reductase domain that provides the necessary electron equivalents from NADPH to reduce the heme cofactor and drive catalysis. The presence of a fused reductase domain in P450 BM3 creates a self-sufficient monooxygenase, obviating the need for exogenous accessory proteins for oxygen activation (see, id.). It has been shown that the N-terminal heme domain can be isolated as an individual, well-folded, soluble protein that retains activity in the presence of hydrogen peroxide as a terminal oxidant under appropriate conditions (P. C. Cirino et al., Angew. Chem., Int. Ed. 42, 3299 (2003)).

In some embodiments, the invention provides reaction mixtures wherein the cytochrome P450 BM3 enzyme variant comprises the C400H mutation and at least two mutations selected from V78M, L181V, and L437W. In some embodiments, the cytochrome P450 BM3 enzyme variant comprises the C400H, V78M, L181V, and L437W mutations. In some embodiments, the cytochrome P450 BM3 enzyme variant further comprises a T268A mutation. In particular embodiments, the cytochrome P450 BM3 enzyme variant comprises or consists of the C400H, V78M, L181V, T268A, and L437W mutations.

One of skill in the art will appreciate that other cytochrome P450 enzyme variants can be used in the methods of the invention. Typically, the P450 BM3 enzyme variant comprises or consists of the heme domain of the wild-type P450 BM3 enzyme sequence (e.g., amino acids 1-463 of SEQ ID NO:1) and at least one of the mutations described herein. In some embodiments, the P450 BM3 enzyme variant comprises or consists of a fragment of the heme domain of the wild-type P450 BM3 enzyme sequence (SEQ ID NO:1), wherein the fragment contains the mutations described herein and is capable of carrying out the cyclopropanation reactions of the invention. In some instances, the fragment includes the heme axial ligand and at least one, two, three, four, or five of the active site residues.

In certain other instances, the cytochrome P450 BM3 enzyme variant is a natural variant thereof as described, e.g., in J. Y. Kang et al., AMB Express 1:1 (2011), wherein the natural variants are divergent in amino acid sequence from the wild-type cytochrome P450 BM3 enzyme sequence (SEQ ID NO:1) by up to about 5% (e.g., SEQ ID NOS:2-11). In such instances, the cytochrome P450 BM3 enzyme variants contain one or more mutations at the residues analogous to C400, V78, L181, T268, and L437 in SEQ ID NO:1.

In certain embodiments, the conserved cysteine residue in a naturally-occurring cytochrome P450 BM3 enzyme variant of interest that serves as the heme axial ligand and is attached to the iron in protoporphyrin IX can be identified by locating the segment of the DNA sequence in the corresponding cytochrome P450 gene which encodes the conserved cysteine residue. In some instances, this DNA segment is identified through detailed mutagenesis studies in a conserved region of the protein (see, e.g., Shimizu et al., Biochemistry 27, 4138-4141, 1988). In other instances, the conserved cysteine is identified through crystallographic study (see, e.g., Poulos et al., J. Mol. Biol 195:687-700, 1987). Mutation of the conserved cysteine residue to histidine can be conducted according to known techniques, as can the introduction of the other mutations described herein.

In situations where detailed mutagenesis studies and crystallographic data are not available for a naturally-occurring cytochrome P450 BM3 enzyme variant of interest, the axial ligand may be identified through phylogenetic study. Due to the similarities in amino acid sequence between cytochrome P450 BM3 enzyme variants, standard protein alignment algorithms may show a phylogenetic similarity between a cytochrome P450 BM3 enzyme variant for which crystallographic or mutagenesis data exist and a new cytochrome P450 BM3 enzyme variant for which such data do not exist. Thus, the polypeptide sequences of the present invention for which the heme axial ligand is known can be used as a “query sequence” to perform a search against a specific new cytochrome P450 BM3 enzyme variant of interest or a database comprising cytochrome P450 BM3 enzyme variant sequences to identify the heme axial ligand. Such analyses can be performed using the BLAST programs (see, e.g., Altschul et al., J Mol Biol. 215(3):403-10(1990)). Software for performing BLAST analyses publicly available through the National Center for Biotechnology Information (http://ncbi.nlm.nih.gov). BLASTP is used for amino acid sequences. Mutation of the conserved cysteine residue to histidine can be conducted according to known techniques, as can the introduction of the other mutations described herein.

Exemplary parameters for performing amino acid sequence alignments to identify the heme axial ligand in a P450 enzyme of interest using the BLASTP algorithm include E value=10, word size=3, Matrix=Blosum62, Gap opening=11, gap extension=1, and conditional compositional score matrix adjustment. Those skilled in the art will know what modifications can be made to the above parameters, e.g., to either increase or decrease the stringency of the comparison and/or to determine the relatedness of two or more sequences.

TABLE 1
Cytochrome P450 BM3 enzyme variants

| Species | Cyp No. | Accession No. | SEQ ID NO |
|---|---|---|---|
| Bacillus megaterium | 102A1 | ADA57069 | 2 |
| Bacillus megaterium | 102A1 | ADA57068 | 3 |
| Bacillus megaterium | 102A1 | ADA57062 | 4 |
| Bacillus megaterium | 102A1 | ADA57061 | 5 |
| Bacillus megaterium | 102A1 | ADA57059 | 6 |
| Bacillus megaterium | 102A1 | ADA57058 | 7 |
| Bacillus megaterium | 102A1 | ADA57055 | 8 |
| Bacillus megaterium | 102A1 | ACZ37122 | 9 |
| Bacillus megaterium | 102A1 | ADA57057 | 10 |
| Bacillus megaterium | 102A1 | ADA57056 | 11 |

In other embodiments, the P450 BM3 enzyme variant further comprises at least one or more (e.g., at least two, three, four, five, six, seven, eight, nine, ten, eleven, or all twelve) of the following amino acid substitutions in SEQ ID NO:1: F87V, P142S, T1751, A184V, S226R, H236Q, E252G, T268A, A290V, L353V, I366V, and E442K. In other instances, the P450 BM3 enzyme variant comprises all twelve of these amino acid substitutions (i.e., F87V, P142S, T1751, A184V, S226R, H236Q, E252G, T268A, A290V, L353V, 1366V, and E442K) in combination with the C400H, V78M, L181V, and/or L437M mutations in SEQ ID NO:1. In some embodiments, the P450 BM3 enzyme variant further comprises at least one or more (e.g., at least two, or all three) of the following amino acid substitutions in SEQ ID NO:1: I263A, A328G, and a T438 mutation. In certain instances, the T438 mutation is T438A, T438S, or T438P.

In other embodiments, the P450 BM3 enzyme variant further comprises one, two, or three) active site alanine substitutions in the active site of SEQ ID NO:1. In certain instances, the active site alanine substitutions are selected from L75A, M177A, I263A, and combinations thereof

In further embodiments, the P450 BM3 enzyme variant further comprises at least one or more (e.g., at least about 2, 3, 4, 5, 6, 7, 8, 9, 10, 11, 12, 13, 14, 15, 16, 17, 18, 19, 20, 21, or 22) of the following amino acid substitutions in SEQ ID NO:1: R47C, L52I, I58V, L75R, F81 (e.g., F81L, F81W), A82 (e.g., A82S, A82F, A82G, A82T, etc.), F87A, K94I, I94K, H100R, S106R, F107L, A135S, F1621, A197V, F205C, N239H, R255S, S274T, L3241, A328V, V340M, and K434E in combination with the mutations described above.

In particular embodiments, cytochrome P450 BM3 variants with at least one or more amino acid mutations catalyze cyclopropanation reactions efficiently, displaying increased total turnover numbers and demonstrating highly regio- and/or enantioselective product formation compared to the wild-type enzyme. For example, the cytochrome P450 BM3 enzyme variants of the present invention can be cis-selective catalysts or trans-selective catalysts. Cis-selective catalysts typically demonstrate diastereomeric ratios at least comparable to wild-type P450 BM3, e.g., at least 37:63 cis:trans, at least 50:50 cis:trans, at least 60:40 cis:trans, or at least 95:5 cis:trans. Trans-selective catalysts typically demonstrate diastereomeric ratios at least comparable to wild-type P450 BM3, e.g., at least 37:63 cis:trans, at least 20:80 cis:trans, or at least 1:99 cis:trans. Mutations for improving cis-selectivity or trans-selectivity are usually isolated to the heme domain of a P450 BM3 enzyme variant and are located in various regions of the heme domain structure including the active site and periphery.

In certain embodiments, the present invention also provides cytochrome P450 BM3 enzyme variants that catalyze enantioselective cyclopropanation with enantiomeric excess values of at least 30% (comparable with wild-type P450 BM3), but more preferably at least 80%, and even more preferably at least >95% for preferred product diastereomers.

An enzyme's total turnover number (or TTN) refers to the maximum number of molecules of a substrate that the enzyme can convert before becoming inactivated. In general, the TTN for the cytochrome P450 BM3 enzyme variants of the invention range from about 1 to about 100,000 or higher. For example, the TTN can be from about 1 to about 1,000, or from about 1,000 to about 10,000, or from about 10,000 to about 100,000, or from about 50,000 to about 100,000, or at least about 100,000. In particular embodiments, the TTN can be from about 100 to about 10,000, or from about 10,000 to about 50,000, or from about 5,000 to about 10,000, or from about 1,000 to about 5,000, or from about 100 to about 1,000, or from about 250 to about 1,000, or from about 100 to about 500, or at least about 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, or more. In certain embodiments, the cytochrome P450 BM3 enzyme variants of the present invention have higher TTNs compared to the wild-type sequences. In some instances, the cytochrome P450 BM3 enzyme variants have TTNs greater than about 100 (e.g., at least about 100, 150, 200, 250, 300, 325, 350, 400, 450, 500, or more) in carrying out in vitro cyclopropanation reactions. In other instances, the cytochrome P450 BM3 enzyme variants have TTNs greater than about 1000 (e.g., at least about 1000, 2500, 5000, 10,000, 25,000, 50,000, 75,000, 100,000, or more) in carrying out in vivo whole cell cyclopropanation reactions.

When whole cells expressing a cytochrome P450 BM3 enzyme variant are used to carry out a cyclopropanation reaction, the turnover can be expressed as the amount of substrate that is converted to product by a given amount of cellular material. In general, in vivo cyclopropanation reactions exhibit turnovers from at least about 0.01 to at least about 10 mmol·g_{cdw}⁻¹, wherein g_{cdw} is the mass of cell dry weight in grams. For example, the turnover can be from about 0.1 to about 10 mmol·g_{cdw}⁻¹, or from about 1 to about 10 mmol·g_{cdw}⁻¹, or from about 5 to about 10 mmol·g_{cdw}⁻¹, or from about 0.01 to about 1 mmol·g_{cdw}⁻¹, or from about 0.01 to about 0.1 mmol·g_{cdw}⁻¹, or from about 0.1 to about 1 mmol·g_{cdw}⁻¹, or greater than 1 mmol·g_{cdw}⁻¹. The turnover can be about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10 mmol·g_{cdw}¹.

When whole cells expressing a cytochrome P450 BM3 enzyme variant are used to carry out a cyclopropanation reaction, the activity can further be expressed as a specific productivity, e.g., concentration of product formed by a given concentration of cellular material per unit time, e.g., in g/L of product per g/L of cellular material per hour (g g_{cdw}⁻¹ h⁻¹). In general, in vivo cyclopropanation reactions exhibit specific productivities from at least about 0.01 to at least about 0.5 g·g_{cdw}⁻¹ h⁻¹, wherein g_{cdw} is the mass of cell dry weight in grams. For example, the specific productivity can be from about 0.01 to about 0.1 g g_{cdw}⁻¹ h⁻¹, or from about 0.1 to about 0.5 g g_{cdw}⁻¹ h⁻¹, or greater than 0.5 g g_{cdw}⁻¹ h⁻¹. The specific productivity can be about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, or about 0.5 g g_{cdw}⁻¹ h⁻¹.

In certain embodiments, mutations can be introduced into the target gene using standard cloning techniques (e.g., site-directed mutagenesis) or by gene synthesis to produce the cytochrome P450 BM3 enzyme variants of the present invention. The mutated gene can be expressed in a host cell (e.g., bacterial cell) using an expression vector under the control of an inducible promoter or by means of chromosomal integration under the control of a constitutive promoter. Cyclopropanation activity can be screened in vivo or in vitro by following product formation by GC or HPLC as described herein.

The expression vector comprising a nucleic acid sequence that encodes a cytochrome P450 BM3 enzyme variant of the invention can be a viral vector, a plasmid, a phage, a phagemid, a cosmid, a fosmid, a bacteriophage (e.g., a bacteriophage P1-derived vector (PAC)), a baculovirus vector, a yeast plasmid, or an artificial chromosome (e.g., bacterial artificial chromosome (BAC), a yeast artificial chromosome (YAC), a mammalian artificial chromosome (MAC), and human artificial chromosome (HAC)). Expression vectors can include chromosomal, non-chromosomal, and synthetic DNA sequences. Equivalent expression vectors to those described herein are known in the art and will be apparent to the ordinarily skilled artisan.

The expression vector can include a nucleic acid sequence encoding a cytochrome P450 BM3 enzyme variant that is operably linked to a promoter, wherein the promoter comprises a viral, bacterial, archaeal, fungal, insect, or mammalian promoter. In certain embodiments, the promoter is a constitutive promoter. In some embodiments, the promoter is an inducible promoter. In other embodiments, the promoter is a tissue-specific promoter or an environmentally regulated or a developmentally regulated promoter.

It is understood that affinity tags may be added to the N- and/or C-terminus of a cytochrome P450 BM3 enzyme variant expressed using an expression vector to facilitate protein purification. Non-limiting examples of affinity tags include metal binding tags such as His6-tags and other tags such as glutathione S-transferase (GST).

Non-limiting expression vectors for use in bacterial host cells include pCWori, pET vectors such as pET22 (EMD Millipore), pBR322 (ATCC37017), pQE™ vectors (Qiagen), pBluescript™ vectors (Stratagene), pNH vectors, lambda-ZAP vectors (Stratagene); ptrc99a, pKK223-3, pDR540, pRIT2T (Pharmacia), pRSET, pCR-TOPO vectors, pET vectors, pSyn_{—}1 vectors, pChlamy_{—}1 vectors (Life Technologies, Carlsbad, Calif.), pGEM1 (Promega, Madison, Wis.), and pMAL (New England Biolabs, Ipswich, Mass.). Non-limiting examples of expression vectors for use in eukaryotic host cells include pXT1, pSG5 (Stratagene), pSVK3, pBPV, pMSG, pSVLSV40 (Pharmacia), pcDNA3.3, pcDNA4/TO, pcDNA6/TR, pLenti6/TR, pMT vectors (Life Technologies), pKLAC1 vectors, pKLAC2 vectors (New England Biolabs), pQE™ vectors (Qiagen), BacPak baculoviral vectors, pAdeno-X™ adenoviral vectors (Clontech), and pBABE retroviral vectors. Any other vector may be used as long as it is replicable and viable in the host cell.

The host cell can be a bacterial cell, an archaeal cell, a fungal cell, a yeast cell, an insect cell, or a mammalian cell.

Suitable bacterial host cells include, but are not limited to, BL21 E. coli, DE3 strain E. coli, E. coli M15, DH5α, DH10β, HB101, T7 Express Competent E. coli (NEB), B. subtilis cells, Pseudomonas fluorescens cells, and cyanobacterial cells such as Chlamydomonas reinhardtii cells and Synechococcus elongates cells. Non-limiting examples of archaeal host cells include Pyrococcus furiosus, Metallosphera sedula, Thermococcus litoralis, Methanobacterium thermoautotrophicum, Methanococcus jannaschii, Pyrococcus abyssi, Sulfolobus solfataricus, Pyrococcus woesei, Sulfolobus shibatae, and variants thereof. Fungal host cells include, but are not limited to, yeast cells from the genera Saccharomyces (e.g., S. cerevisiae), Pichia (P. pastoris), Kluyveromyces (e.g., K. lactis), Hansenula and Yarrowia, and filamentous fungal cells from the genera Aspergillus, Trichoderma, and Myceliophthora. Suitable insect host cells include, but are not limited to, Sf9 cells from Spodoptera frugiperda, Sf21 cells from Spodoptera frugiperda, Hi-Five cells, BTI-TN-5B1-4 Trichophusia ni cells, and Schneider 2 (S2) cells and Schneider 3 (S3) cells from Drosophila melanogaster. Non-limiting examples of mammalian host cells include HEK293 cells, HeLa cells, CHO cells, COS cells, Jurkat cells, NS0 hybridoma cells, baby hamster kidney (BHK) cells, MDCK cells, NIH-3T3 fibroblast cells, and any other immortalized cell line derived from a mammalian cell.

In certain embodiments, the present invention provides cytochrome P450 BM3 enzyme variants that are active cyclopropanation catalysts inside living cells. As a non-limiting example, bacterial cells (e.g., E. coli) can be used as whole cell catalysts for the in vivo cyclopropanation reactions of the present invention. In some embodiments, whole cell catalysts containing the cytochrome P450 BM3 enzyme variants are found to significantly enhance the total turnover number (TTN) compared to in vitro reactions using isolated P450 enzymes.

In certain embodiments, the present invention provides amino acid substitutions that efficiently remove monooxygenation chemistry from cytochrome P450 BM3 enzyme variants. This system permits selective enzyme-driven cyclopropanation chemistry without competing side reactions mediated by native cytochrome P450 BM3 enzyme catalysis. The invention also provides cytochrome P450 BM3-mediated catalysis that is competent for cyclopropanation chemistry but not able to carry out traditional P450-mediated monooxygenation reactions The present invention further provides a compatible reducing agent for orthogonal P450 cyclopropanation catalysis that includes, but is not limited to, NAD(P)H or sodium dithionite.

B. Compounds

The methods of the invention can be used to provide a number of cyclopropanation products. The cyclopropanation products include several classes of compounds including, but not limited to, commodity and fine chemicals, flavors and scents, insecticides, and active ingredients in pharmaceutical compositions. The cyclopropanation products can also serve as starting materials or intermediates for the synthesis of compounds belonging to these and other classes.

In some embodiments, the cyclopropanation product is a compound according to Formula 1:

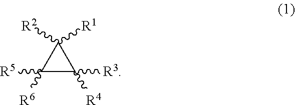

For compounds of Formula 1, R¹ is independently selected from H, optionally substituted C₁₋₁₈ alkyl, optionally substituted C₆₋₁₀ aryl, optionally substituted 6- to 10-membered heteroaryl, halo, cyano, C(O)OR^{1a}, C(O)N(R⁷)₂, C(O)R⁸, C(O)C(O)OR⁸, and Si(R⁸)₃; and R² is independently selected from H, optionally substituted C₁₋₁₈ alkyl, optionally substituted C₆₋₁₀ aryl, optionally substituted 6- to 10-membered heteroaryl, halo, cyano, C(O)OR^{2a}, C(O)N(R⁷)₂, C(O)R⁸, C(O)C(O)OR⁸, and Si(R⁸)₃. R^{1a} and R^{2a} H, optionally substituted C₁₋₁₈ alkyl and -L-R^{C}.

When the moiety -L-R^{C} is present, L is selected from a bond, —C(R^{L})₂—, and —NR^{L}—C(R^{L})₂—. Each R^{L} is independently selected from H, C₁₋₆ alkyl, halo, —CN, and —SO₂, and each R^{C} is selected from optionally substituted C₆₋₁₀ aryl, optionally substituted 6- to 10-membered heteroraryl, and optionally substituted 6- to 10-membered heterocyclyl.

For compounds of Formula 1, R³, R⁴, R⁵, and R⁶ are independently selected from H, C₁₋₁₈ alkyl, C₂₋₁₈ alkenyl, C₂₋₁₈ alkynyl, optionally substituted C₆₋₁₀ aryl, optionally substituted 5- to 10-membered heteroaryl, optionally substituted C₁-C₆ alkoxy, halo, hydroxy, cyano, C(O)N(R⁷)₂, NR⁷C(O)R⁸, C(O)R⁸, C(O)OR⁸, and N(R⁹)₂. Each R⁷ and R⁸ is independently selected from H, optionally substituted C₁₋₁₈ alkyl, 2- to 18-membered heteroalkyl, optionally substituted C₂₋₁₂ alkenyl, hydroxyl, C₁₋₁₈ alkoxy, C₃₋₈ cycloalkyl, C₁₋₁₈ fluoroalkyl, optionally substituted C₆₋₁₀ aryl, and optionally substituted 5- to 10-membered heteroaryl. Alternatively, two R⁷ moieties are taken together with the nitrogen atom to which they are bonded to form optionally substituted 5- to 10-membered heterocyclyl or optionally substituted 5- to 10-membered heteroaryl. Each R⁹ is independently selected from H, optionally substituted C₆₋₁₀ aryl, and optionally substituted 6- to 10-membered heteroaryl. Alternatively, two R⁹ moieties, together with the nitrogen atom to which they are attached, can form 6- to 18-membered heterocyclyl.

Alternatively, R³ forms an optionally substituted 3- to 18-membered ring with R⁴, or R⁵ forms an optionally substituted 3 to 18-membered ring with R⁶. R³ or R⁴ can also form a double bond with R⁵ or R⁶. R³ or R⁴ forms an optionally substituted 5- to 6-membered ring with R⁵ or R⁶.

In some embodiments, the invention provides reaction mixtures as described above, wherein the olefinic substrate contains one or more electron withdrawing groups. In some embodiments, the olefinic substrate is an acrylamide compound according to Formula I:

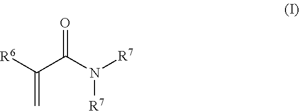

wherein:
each R⁷ is independently selected from H, optionally substituted C₁₋₁₈ alkyl, 2- to 18-membered heteroalkyl, hydroxyl, C₁₋₁₈ alkoxy, C₃₋₈ cycloalkyl, C₁₋₁₈ fluoroalkyl, optionally substituted C₆₋₁₀ aryl, optionally substituted 5- to 10-membered heteroaryl, or are taken together with the nitrogen atom to which they are bonded to form optionally substituted 5- to 10-membered heterocyclyl or optionally substituted 5- to 10-membered heteroaryl; and
R⁶ is selected from optionally substituted C₆₋₁₀ aryl and optionally substituted 5- to 10-membered heteroaryl.

In some embodiments, the olefinic substrate is selected from:

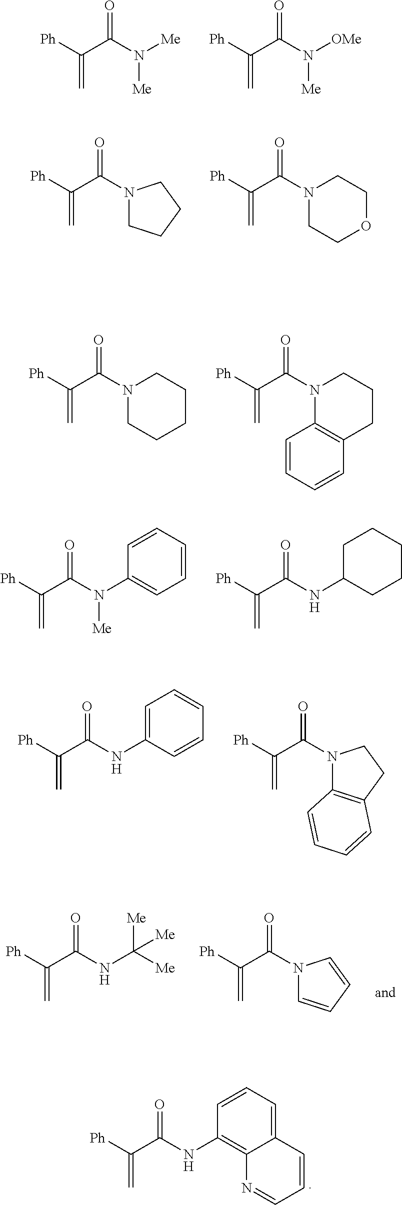

In some embodiments, the invention provides a reaction mixture wherein the olefinic substrate is an acrylate compound according to Formula II:

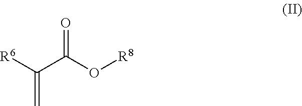

wherein
R⁸ is independently selected from H, optionally substituted C₁₋₁₈ alkyl, 2- to 18-membered heteroalkyl, C₃₋₈ cycloalkyl, C₁₋₁₈ fluoroalkyl, optionally substituted C₆₋₁₀ aryl, and optionally substituted 5- to 10-membered heteroaryl; and
R⁶ is selected from optionally substituted C₆₋₁₀ aryl and optionally substituted 5- to 10-membered heteroaryl.

In some embodiments, the olefinic substrate is selected from:

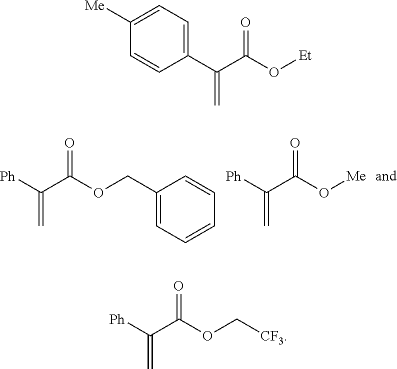

Any suitable carbene precursor can be used in the methods and reaction mixtures of the invention. In some embodiments, the carbene precursor is a diazo reagent. In some embodiments, the diazo reagent is selected from an α-diazoester, an α-diazoamide, an α-diazonitrile, an α-diazoketone, an α-diazoaldehyde, and an α-diazosilane. In some embodiments, the diazo reagent is selected from:

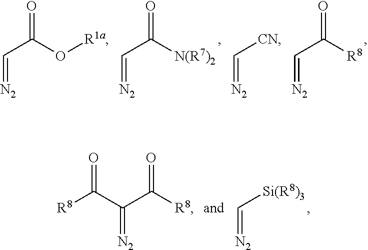

wherein
R^{1a} is selected from H and optionally substituted C₁₋₆ alkyl; and each R⁷ and each R⁸ is independently selected from H, optionally substituted C₁₋₁₂ alkyl, optionally substituted C₂₋₁₂ alkenyl, and optionally substituted C₆₋₁₀ aryl.

A number of other compounds can be synthesized via processes that include a cyclopropanation product. Such processes are generalized in Scheme 2 showing the enzyme-catalyzed formation of a cyclopropanation product from an olefinic substrate and a diazo reagent, followed by chemical conversion of to a final product such as a pharmaceutical agent.

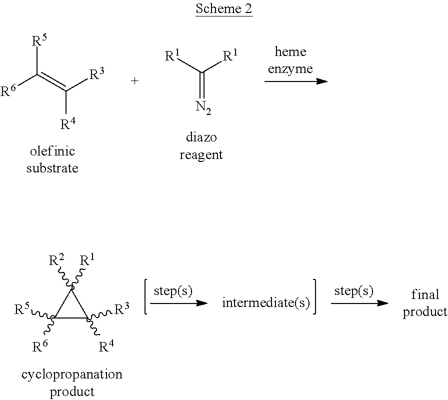

Depending on the particular final product, the process can include conversion of the cyclopropanation product to one or more synthetic intermediates prior to preparation of the final product. Non-limiting examples of cyclopropanation products useful in such processes are summarized in Table 2.

TABLE 2
Cyclopropanation for synthesis of intermediates en route to biologically active
compounds.

| | | Cyclopropanation | |
|---|---|---|---|
| Olefinic Substrate | Diazo Reagent | Product/Intermediate | Final Product |
| 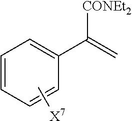 | 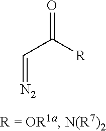 | 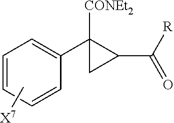 | milnacipran |
| 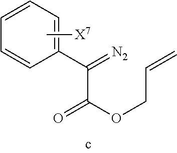 | | 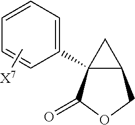 | milnacipran |
| 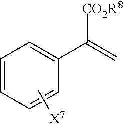 | 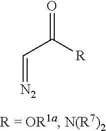 | 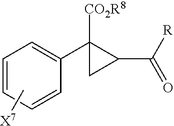 | milnacipran; bicifidine; 1-(3,4- dichloro- phenyl)-3- azabicyclo [3.1.0]hexane |
| 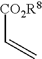 | 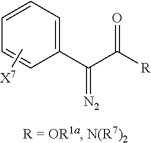 | 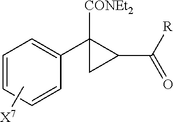 | milnacipran; bicifidine; 1-(3,4- dichloro- phenyl)-3- azabicyclo [3.1.0]hexane |
| 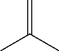 | 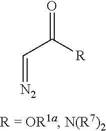 | 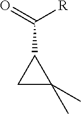 | cilastain |
| 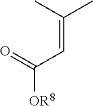 | 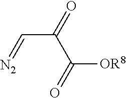 | 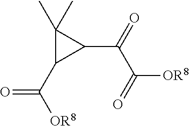 | boceprevir |
| 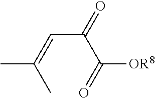 | 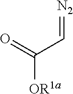 | 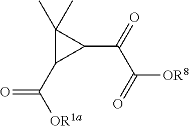 | boceprevir |
| 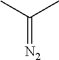 | 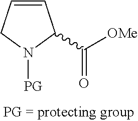 | 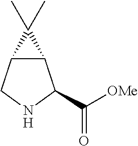 | boceprevir |
| 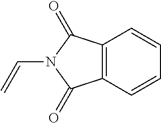 | 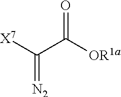 | 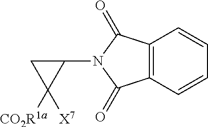 | 1R,2S- fluorocyclo- propyl- amine, sitafloxacin |
| 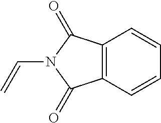 | 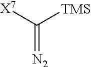 | 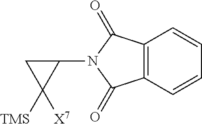 | 1R,2S- fluorocyclo- propyl- amine, sitafloxacin |
| 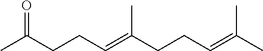 | 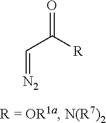 | 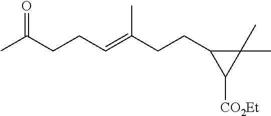 [SMILES: CCOC(=O)C1C(CC/C(C)=C/CCC(C)=O)C1(C)C] | anthoplalone, noranthoplone |
| 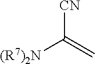 | 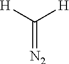 | 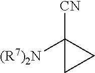 | odanacatib |
| 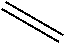 | 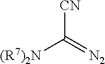 | 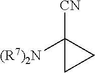 | odanacatib |
| 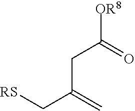 | 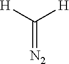 | 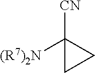 | montekulast |
| 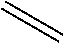 | 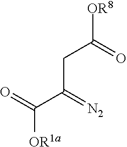 | 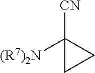 | montekulast |
| 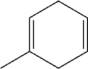 | 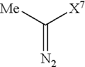 | 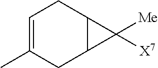 | carene |
| 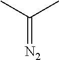 | 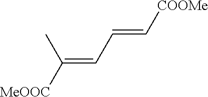 | 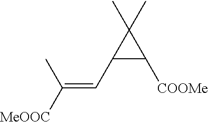 [SMILES: COC(=O)/C(C)=C/C1C(C(=O)OC)C1(C)C] | pyrethrin II |

In some embodiments, the cyclopropanation product is a compound having a structure according to the formula:

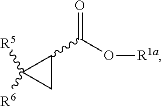

wherein R^{1a} is optionally substituted C₁₋₆ alkyl, and R⁵ and R⁶ are independently selected from H, optionally substituted C₁₋₆ alkyl, optionally substituted C₆₋₁₀ aryl, C(O)N(R⁷)₂, C(O)OR⁸ and NR⁷C(O)R⁸.

In some embodiments, the cyclopropanation product has the structure selected from:

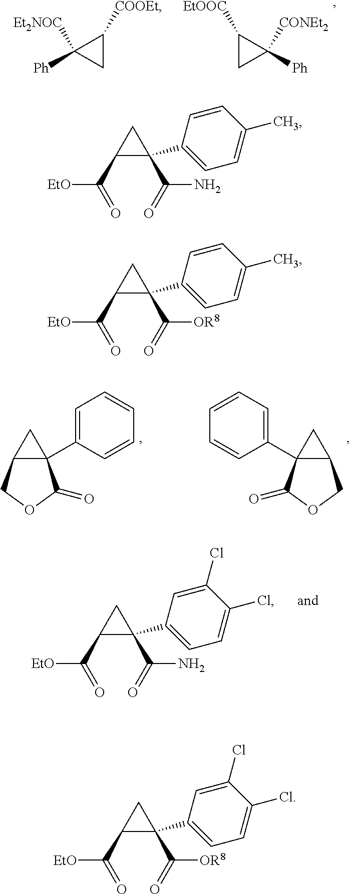

In such embodiments, the methods of the invention can include converting the cyclopropanation product to a compound selected from milcanipran, levomilnacipran, bicifadine, and 1-(3,4-dichlorophenyl)-3-azabicyclo[3.1.0]hexane.

The methods of the invention can be used to prepare several different types of compounds having cyclopropane functional groups. The compounds include, but are not limited to, pharmaceutical agents having chiral cyclopropane moieties, pharmaceutical agents having achiral cyclopropane moieties, insecticides, plant hormones, flavors, scents, and fatty acids.

In some embodiments, the methods of the invention are used to prepare a compound selected from:

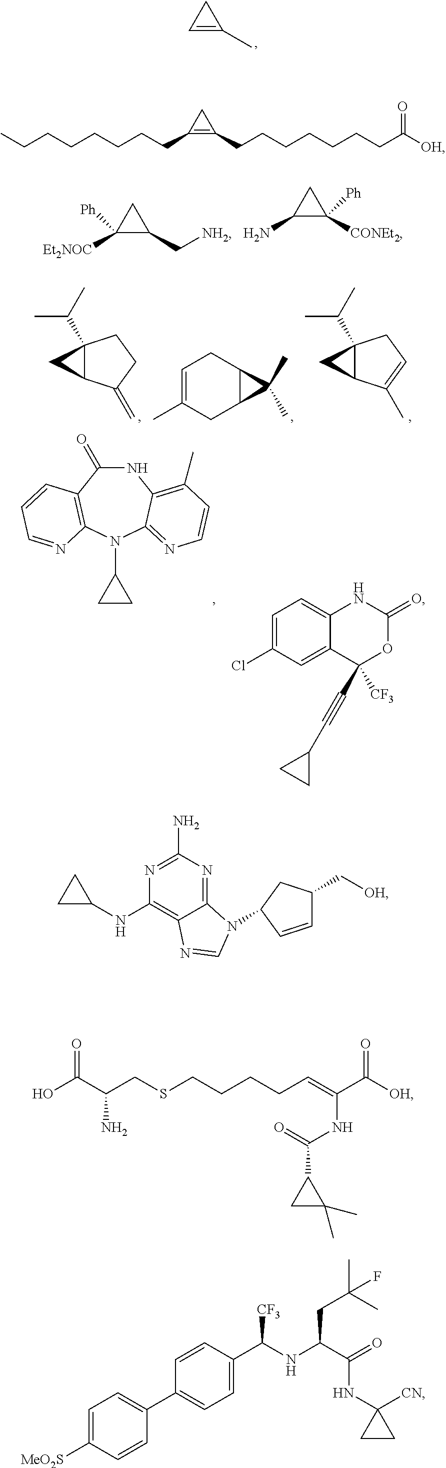

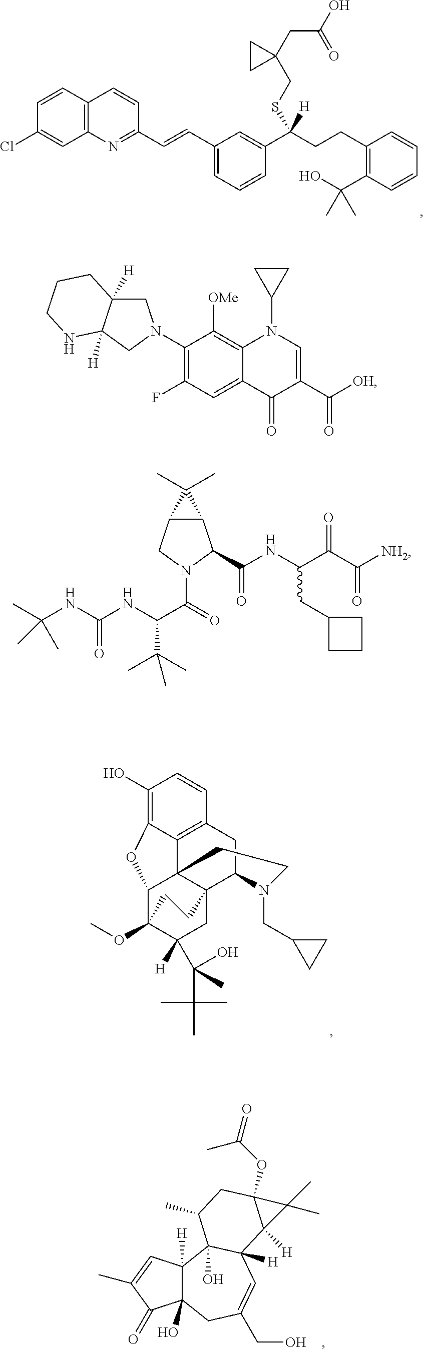

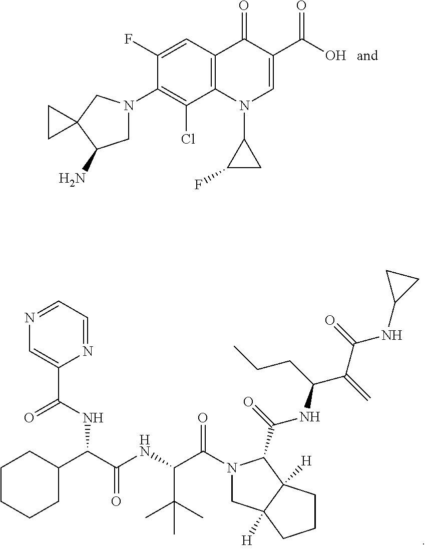 [SMILES: N[C@@H]1CN(c2c(F)cc3c(=O)c(C(=O)O)cn(C4C[C@@H]4F)c3c2Cl)CC12CC2 ; C=C(C(=O)NC1CC1)[C@H](CCC)NC(=O)[C@@H]1[C@H]2CCC[C@H]2CN1C(=O)[C@@H](NC(=O)[C@@H](NC(=O)c1cnccn1)C1CCCCC1)C(C)(C)C]

Synthesis of prostratin, a protein kinase C activator, is shown as a non-limiting example in Scheme 3. The prostratin cyclopropane moiety can be installed by heme enzyme-catalyzed intramolecular or intermolecular cyclopropanation reactions.

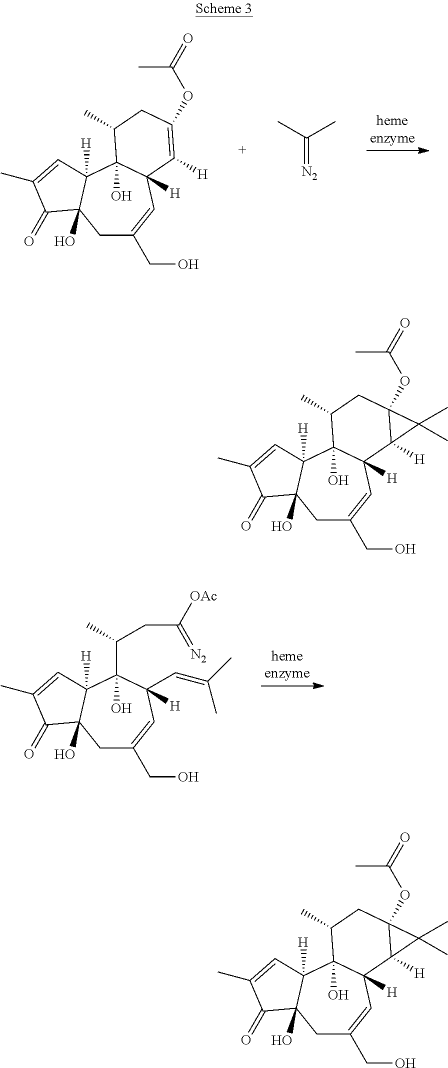

Some embodiments of the invention provide a method as described above, wherein the olefinic substrate is selected from an alkene, a cycloalkane, and an arylalkene. In some embodiments, the olefinic substrate is an arylalkene. In some embodiments, the arylalkene is a styrene.

In some embodiments, the styrene has the formula:

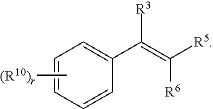

R³ is selected from the H, optionally substituted C₁-C₆ alkyl, optionally substituted C₁-C₆ alkoxy, C(O)N(R⁷)₂, C(O)OR⁸, N(R⁹)₂, halo, hydroxy, and cyano. R⁵ and R⁶ are independently selected from H, optionally substituted C₁₋₆ alkyl, and halo. R¹⁰ is selected from optionally substituted C₁-C₆ alkyl, optionally substituted C₁-C₆ alkoxy, halo, and haloalkyl, and the subscript r is an integer from 0 to 2.

In general, the diazo reagents useful in the methods of the invention have the structure:

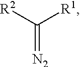

wherein R¹ and R² are defined as for the cyclopropanation products. Any diazoreagent can be added to the reaction as a reagent itself, or the diazoreagent can be prepared in situ.

In some embodiments, the diazo reagent is selected from an α-diazoester, an α-diazoamide, an α-diazonitrile, an α-diazoketone, an α-diazoaldehyde, and an α-diazosilane. In some embodiments, the diazo reagent has a formula selected from:

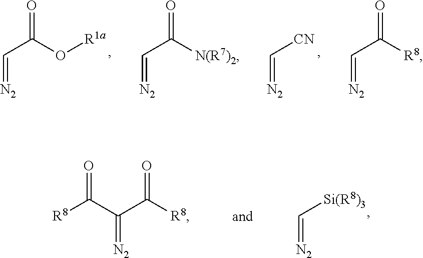

wherein R^{1a} is selected from H and optionally substituted C₁-C₆ alkyl; and each R⁷ and R⁸ is independently selected from H, optionally substituted C₁₋₁₂ alkyl, optionally substituted C₂₋₁₂ alkenyl, and optionally substituted C₆₋₁₀ aryl.

In some embodiments, the diazo reagent is selected from diazomethane, ethyl diazoacetate, and (trimethylsilyl)diazomethane.

In some embodiments, the diazo reagent is an α-diazoester. In some embodiments, the diazo reagent has the formula:

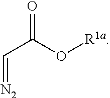

In some embodiments, the cyclopropanation product has a formula selected from:

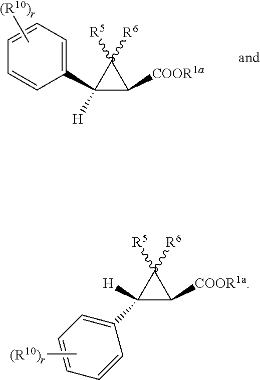

In some embodiments, the cyclopropanation product is a compound of Formula 1 as described above, wherein R¹ is C(O)O-LR^{c}; R² is selected from H and optionally substituted C₆₋₁₀ aryl; and R³, R⁴, R⁵, and R⁶ are independently selected from H, optionally substituted C₁₋₆ alkyl, optionally substituted C₂₋₆ alkenyl, optionally substituted C₂₋₆ alkynyl, optionally substituted C₆₋₁₀ aryl, and halo. Alternatively, R³ can form an optionally substituted 3 to 18-membered ring with R⁴, or R⁵ can form an optionally substituted 3 to 18-membered ring with R⁶. In such embodiments, the cyclopropanation product can be a pyrethroid or a pyrethroid precursor.

In some embodiments, pyrethroids are produced via the methods of the invention. In general, pyrethroids are characterized by an ester core having a structure according to Formula 3:

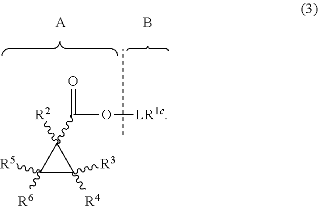

Formula 3 is presented above as a cyclopropyl carboxylate moiety (“A”) esterified with an LR^{1c} moiety (“B”), with R^{1c} defined as for R^{C}. The methods of the invention can be used to prepare pyrethroids and pyrethroid intermediates having a variety of “A” moieties connected to any of a variety of “B” moieties. For example, the pyrethroids can have an “A” moiety selected from:

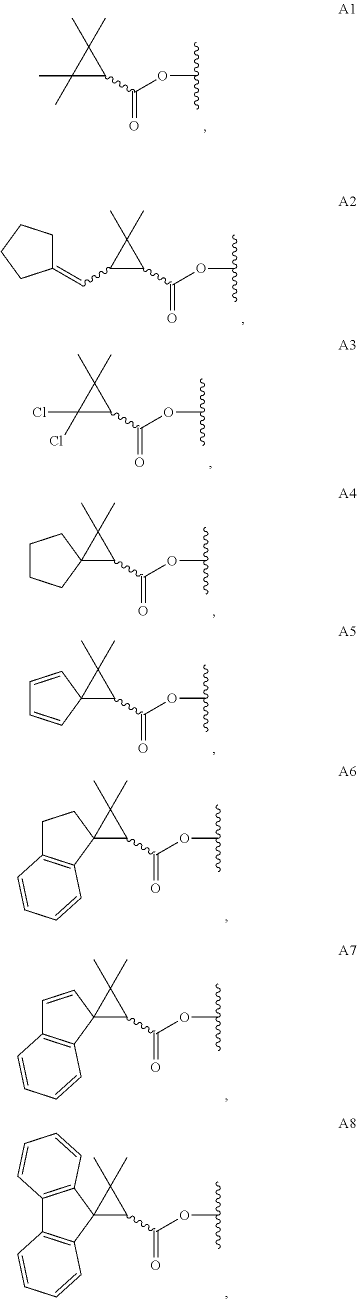

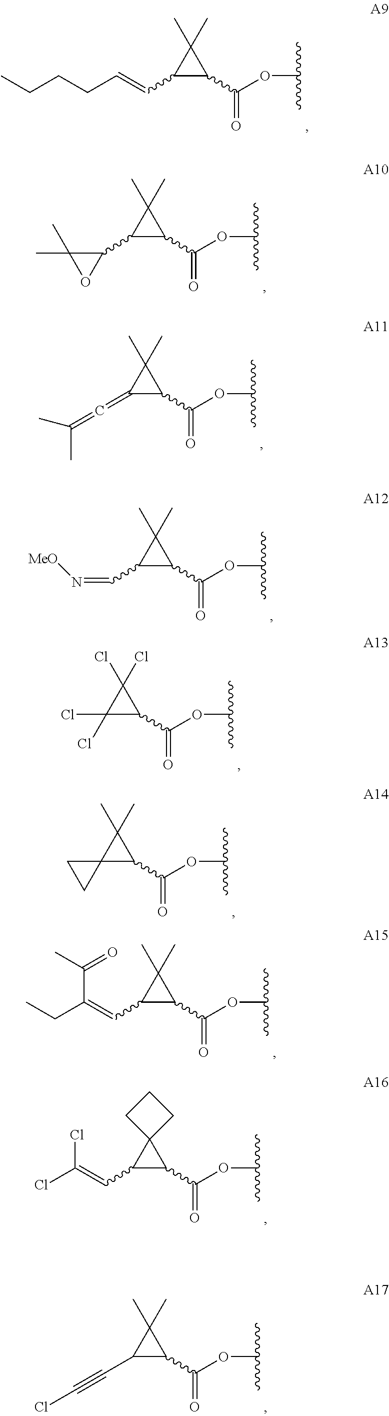

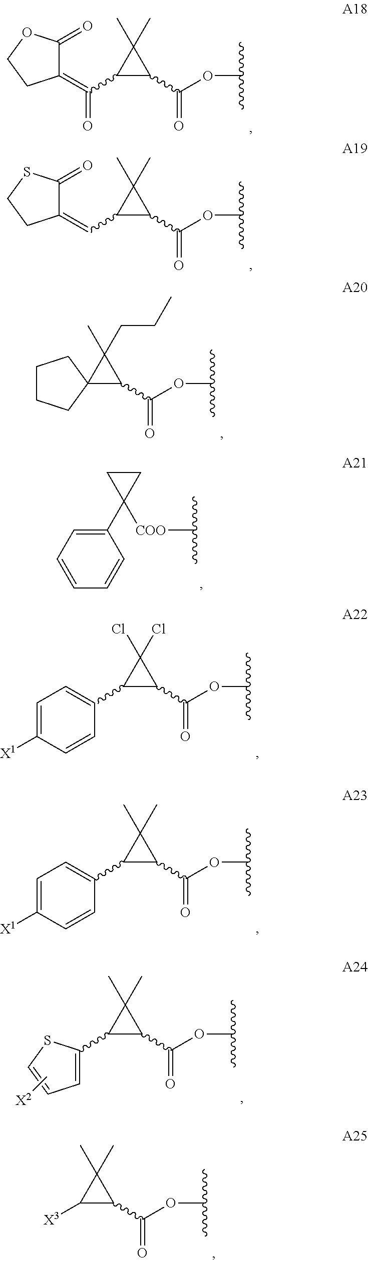

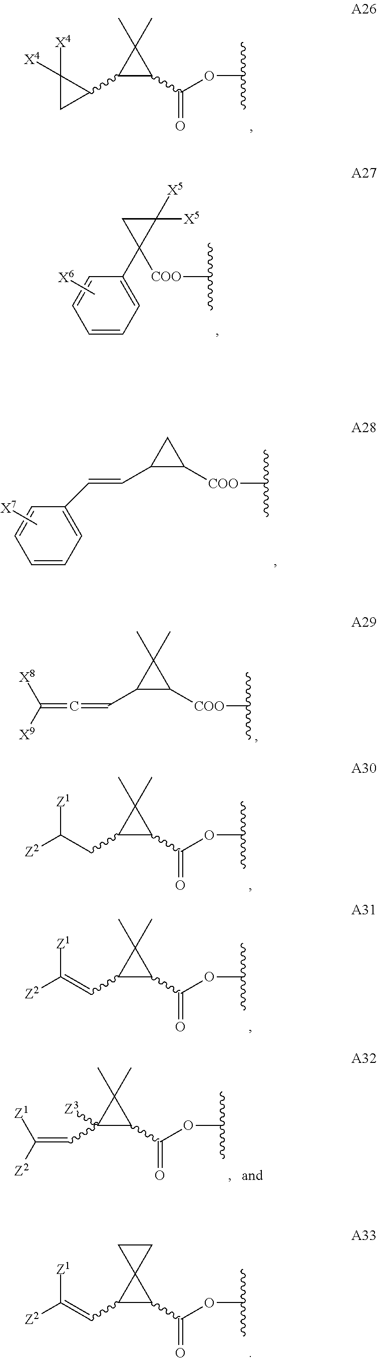

For the A moieties listed above, X¹ is selected from H, optionally substituted C₁₋₆ alkyl, haloC₁₋₆ alkyl, optionally substituted C₁₋₆ alkoxy, haloC₁₋₆ alkyl, optionally substituted C₁₋₆ alkylthio, C₁₋₆ alkylsilyl, halo, and cyano. X² is selected from H, chloro, and methyl. X³ is selected from H, methyl, halo, and CN. Each X⁴ is independently halo. Each X⁵ is independently selected from methyl and halo. X⁶ is selected from halo, optionally substituted C₁₋₆ alkyl, and optionally substituted C₁₋₆ alkoxy. X⁷ is selected from H, methyl, and halo. X⁸ is selected from H, halo, and optionally substituted C₁₋₆ alkyl. X⁹ is selected from H, halo, optionally substituted C₁₋₆ alkyl, C(O)O—(C₁₋₆ alkyl), C(O)—N(C₁₋₆ alkyl)₂, and cyano. Z¹, Z², and Z³ are independently selected from H, halo, optionally substituted C₁₋₆ alkyl, and optionally substituted C₆₋₁₀ aryl, or Z¹ and Z² are taken together to form an optionally substituted 5- to 6-membered cycloalkyl or heterocyclyl group. The wavy line at the right of each structure represents the point of connection between the A moiety and a B moiety.

The pyrethroids can have “B” moieties selected from:

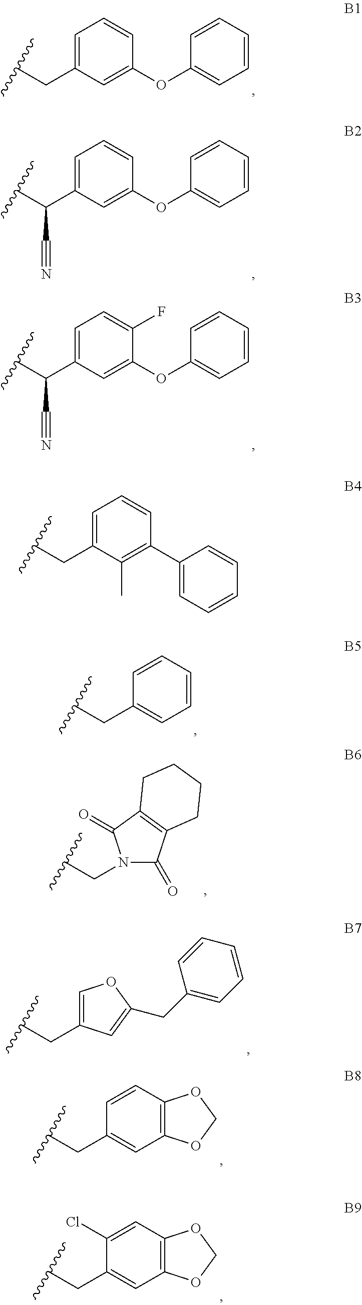

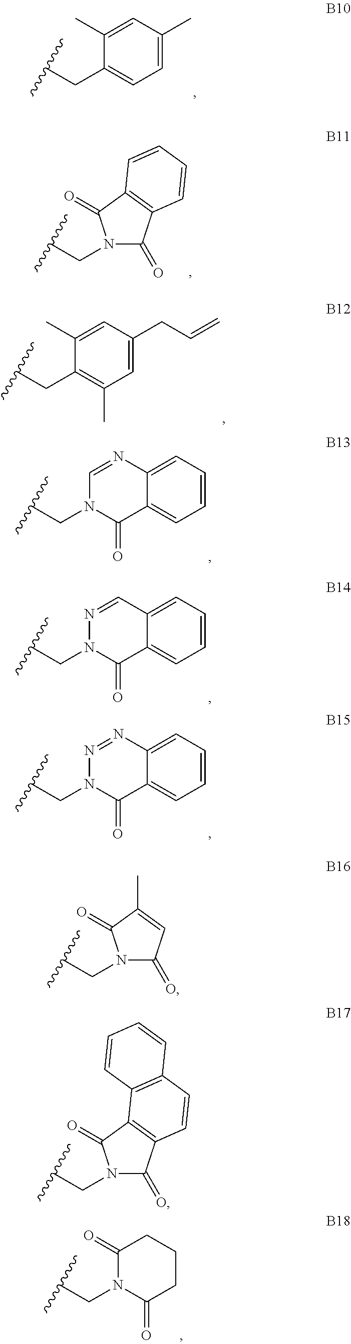

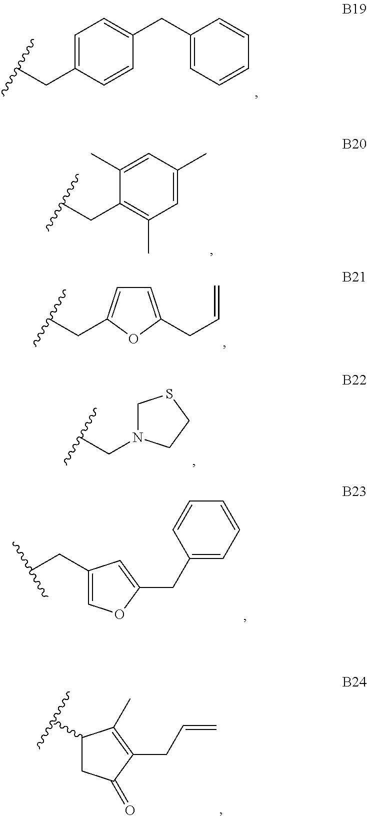

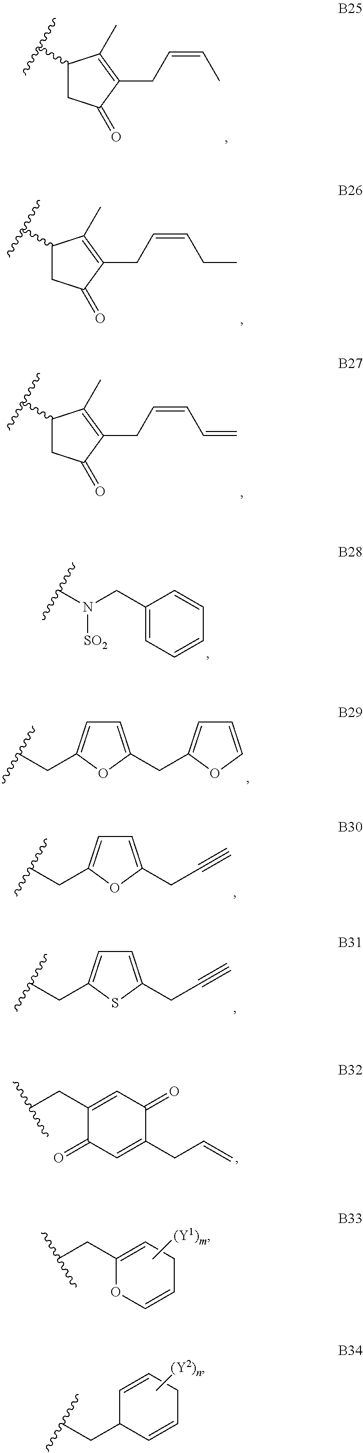

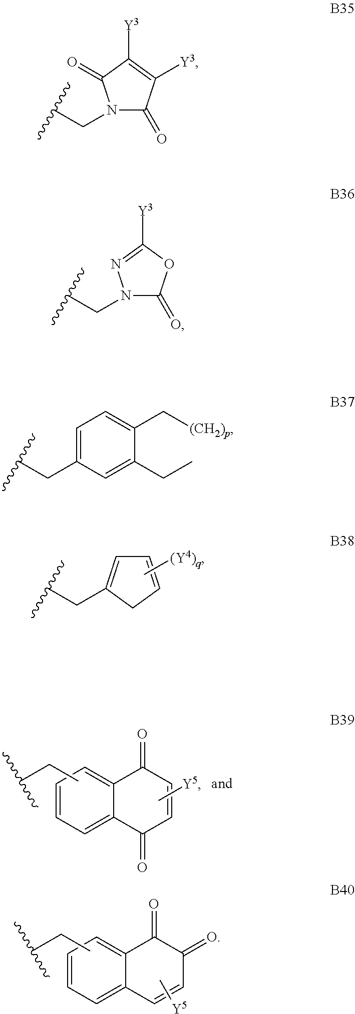

For the B moieties listed above, each Y¹ is independently selected from optionally substituted C₁₋₆ alkyl, optionally substituted C₂₋₆ alkenyl, optionally substituted C₂₋₆ alkynyl, phenyl, and (phenyl)C₁₋₆ alkoxy. Each Y² is independently selected from halo, optionally substituted C₁₋₆ alkyl, optionally substituted C₂₋₆ alkenyl, optionally substituted C₁₋₆ alkoxy, and nitro. Each Y³ is independently optionally substituted C₁₋₆ alkyl. Each Y⁴ is independently selected from optionally substituted C₁₋₆ alkyl, optionally substituted C₂₋₆ alkenyl, optionally substituted C₂₋₆ alkynyl, C₆₋₁₀ aryl-C₁₋₆ alkyl, furfuryl, C₁₋₆ alkoxy, (C₂₋₆ alkenyl)oxy, C₁₋₁₂ acyl, and halo. Y⁵ is selected from optionally substituted C₁₋₆ alkyl, optionally substituted C₁₋₆ alkoxy, and halo. The subscript m is an integer from 1 to 3, the subscript n is an integer from 1 to 5, the subscript p is an integer from 1 to 4, and the subscript q is an integer from 0 to 3. The wavy line at the left of each structure represents the point of connection between the B moiety and an A moiety.

The methods of the invention can be used to prepare pyrethroids having any A moiety joined to any B moiety. A given pyrethroid can have a structure selected from: A1-B1, A2-B1, A3-B1, A4-B1, A5-B1, A6-B1, A7-B1, A8-B1, A9-B1, A10-B1, A11-B1, A12-B1, A13-B1, A14-B1, A15-B1, A16-B1, A17-B1, A18-B1, A19-B1, A20-B1, A21-B1, A22-B1, A23-B1, A24-B1, A25-B1, A26-B1, A27-B1, A28-B1, A29-B1, A30-B1, A31-B1, A32-B1, A33-B1, A1-B2, A2-B2, A3-B2, A4-B2, A5-B2, A6-B2, A7-B2, A8-B2, A9-B2, A10-B2, A11-B2, A12-B2, A13-B2, A14-B2, A15-B2, A16-B2, A17-B2, A18-B2, A19-B2, A20-B2, A21-B2, A22-B2, A23-B2, A24-B2, A25-B2, A26-B2, A27-B2, A28-B2, A29-B2, A30-B2, A31-B2, A32-B2, A33-B2, A1-B3, A2-B3, A3-B3, A4-B3, A5-B3, A6-B3, A7-B3, A8-B3, A9-B3, A10-B3, A11-B3, A12-B3, A13-B3, A14-B3, A15-B3, A16-B3, A17-B3, A18-B3, A19-B3, A20-B3, A21-B3, A22-B3, A23-B3, A24-B3, A25-B3, A26-B3, A27-B3, A28-B3, A29-B3, A30-B3, A31-B3, A32-B3, A33-B3, A1-B4, A2-B4, A3-B4, A4-B4, A5-B4, A6-B4, A7-B4, A8-B4, A9-B4, A10-B4, A11-B4, A12-B4, A13-B4, A14-B4, A15-B4, A16-B4, A17-B4, A18-B4, A19-B4, A20-B4, A21-B4, A22-B4, A23-B4, A24-B4, A25-B4, A26-B4, A27-B4, A28-B4, A29-B4, A30-B4, A31-B4, A32-B4, A33-B4, A1-B5, A2-B5, A3-B5, A4-B5, A5-B5, A6-B5, A7-B5, A8-B5, A9-B5, A10-B5, A11-B5, A12-B5, A13-B5, A14-B5, A15-B5, A16-B5, A17-B5, A18-B5, A19-B5, A20-B5, A21-B5, A22-B5, A23-B5, A24-B5, A25-B5, A26-B5, A27-B5, A28-B5, A29-B5, A30-B5, A31-B5, A32-B5, A33-B5, A1-B6, A2-B6, A3-B6, A4-B6, A5-B6, A6-B6, A7-B6, A8-B6, A9-B6, A10-B6, A11-B6, A12-B6, A13-B6, A14-B6, A15-B6, A16-B6, A17-B6, A18-B6, A19-B6, A20-B6, A21-B6, A22-B6, A23-B6, A24-B6, A25-B6, A26-B6, A27-B6, A28-B6, A29-B6, A30-B6, A31-B6, A32-B6, A33-B6, A1-B7, A2-B7, A3-B7, A4-B7, A5-B7, A6-B7, A7-B7, A8-B7, A9-B7, A10-B7, A11-B7, A12-B7, A13-B7, A14-B7, A15-B7, A16-B7, A17-B7, A18-B7, A19-B7, A20-B7, A21-B7, A22-B7, A23-B7, A24-B7, A25-B7, A26-B7, A27-B7, A28-B7, A29-B7, A30-B7, A31-B7, A32-B7, A33-B7, A1-B8, A2-B8, A3-B8, A4-B8, A5-B8, A6-B8, A7-B8, A8-B8, A9-B8, A10-B8, A11-B8, A12-B8, A13-B8, A14-B8, A15-B8, A16-B8, A17-B8, A18-B8, A19-B8, A20-B8, A21-B8, A22-B8, A23-B8, A24-B8, A25-B8, A26-B8, A27-B8, A28-B8, A29-B8, A30-B8, A31-B8, A32-B8, A33-B8, A1-B9, A2-B9, A3-B9, A4-B9, A5-B9, A6-B9, A7-B9, A8-B9, A9-B9, A10-B9, A11-B9, A12-B9, A13-B9, A14-B9, A15-B9, A16-B9, A17-B9, A18-B9, A19-B9, A20-B9, A21-B9, A22-B9, A23-B9, A24-B9, A25-B9, A26-B9, A27-B9, A28-B9, A29-B9, A30-B9, A31-B9, A32-B9, A33-B9, A1-B10, A2-B10, A3-B10, A4-B10, A5-B10, A6-B10, A7-B10, A8-B10, A9-B10, A10-B10, A11-B10, A12-B10, A13-B10, A14-B10, A15-B10, A16-B10, A17-B10, A18-B10, A19-B10, A20-B10, A21-B10, A22-B10, A23-B10, A24-B10, A25-B10, A26-B10, A27-B10, A28-B10, A29-B10, A30-B10, A31-B10, A32-B10, A33-B10, A1-B11, A2-B11, A3-B11, A4-B11, A5-B11, A6-B11, A7-B11, A8-B11, A9-B11, A10-B11, A11-B11, A12-B11, A13-B11, A14-B11, A15-B11, A16-B11, A17-B11, A18-B11, A19-B11, A20-B11, A21-B11, A22-B11, A23-B11, A24-B11, A25-B11, A26-B11, A27-B11, A28-B11, A29-B11, A30-B11, A31-B11, A32-B11, A33-B11, A1-B12, A2-B12, A3-B12, A4-B12, A5-B12, A6-B12, A7-B12, A8-B12, A9-B12, A10-B12, A11-B12, A12-B12, A13-B12, A14-B12, A15-B12, A16-B12, A17-B12, A18-B12, A19-B12, A20-B12, A21-B12, A22-B12, A23-B12, A24-B12, A25-B12, A26-B12, A27-B12, A28-B12, A29-B12, A30-B12, A31-B12, A32-B12, A33-B12, A1-B13, A2-B13, A3-B13, A4-B13, A5-B13, A6-B13, A7-B13, A8-B13, A9-B13, A10-B13, A11-B13, A12-B13, A13-B13, A14-B13, A15-B13, A16-B13, A17-B13, A18-B13, A19-B13, A20-B13, A21-B13, A22-B13, A23-B13, A24-B13, A25-B13, A26-B13, A27-B13, A28-B13, A29-B13, A30-B13, A31-B13, A32-B13, A33-B13, A1-B14, A2-B14, A3-B14, A4-B14, A5-B14, A6-B14, A7-B14, A8-B14, A9-B14, A10-B14, A11-B14, A12-B14, A13-B14, A14-B14, A15-B14, A16-B14, A17-B14, A18-B14, A19-B14, A20-B14, A21-B14, A22-B14, A23-B14, A24-B14, A25-B14, A26-B14, A27-B14, A28-B14, A29-B14, A30-B14, A31-B14, A32-B14, A33-B14, A1-B15, A2-B15, A3-B15, A4-B15, A5-B15, A6-B15, A7-B15, A8-B15, A9-B15, A10-B15, A11-B15, A12-B15, A13-B15, A14-B15, A15-B15, A16-B15, A17-B15, A18-B15, A19-B15, A20-B15, A21-B15, A22-B15, A23-B15, A24-B15, A25-B15, A26-B15, A27-B15, A28-B15, A29-B15, A30-B15, A31-B15, A32-B15, A33-B15, A1-B16, A2-B16, A3-B16, A4-B16, A5-B16, A6-B16, A7-B16, A8-B16, A9-B16, A10-B16, A11-B16, A12-B16, A13-B16, A14-B16, A15-B16, A16-B16, A17-B16, A18-B16, A19-B16, A20-B16, A21-B16, A22-B16, A23-B16, A24-B16, A25-B16, A26-B16, A27-B16, A28-B16, A29-B16, A30-B16, A31-B16, A32-B16, A33-B16, A1-B17, A2-B17, A3-B17, A4-B17, A5-B17, A6-B17, A7-B17, A8-B17, A9-B17, A10-B17, A11-B17, A12-B17, A13-B17, A14-B17, A15-B17, A16-B17, A17-B17, A18-B17, A19-B17, A20-B17, A21-B17, A22-B17, A23-B17, A24-B17, A25-B17, A26-B17, A27-B17, A28-B17, A29-B17, A30-B17, A31-B17, A32-B17, A33-B17, A1-B18, A2-B18, A3-B18, A4-B18, A5-B18, A6-B18, A7-B18, A8-B18, A9-B18, A10-B18, A11-B18, A12-B18, A13-B18, A14-B18, A15-B18, A16-B18, A17-B18, A18-B18, A19-B18, A20-B18, A21-B18, A22-B18, A23-B18, A24-B18, A25-B18, A26-B18, A27-B18, A28-B18, A29-B18, A30-B18, A31-B18, A32-B18, A33-B18, A1-B19, A2-B19, A3-B19, A4-B19, A5-B19, A6-B19, A7-B19, A8-B19, A9-B19, A10-B19, A11-B19, A12-B19, A13-B19, A14-B19, A15-B19, A16-B19, A17-B19, A18-B19, A19-B19, A20-B19, A21-B19, A22-B19, A23-B19, A24-B19, A25-B19, A26-B19, A27-B19, A28-B19, A29-B19, A30-B19, A31-B19, A32-B19, A33-B19, A1-B20, A2-B20, A3-B20, A4-B20, A5-B20, A6-B20, A7-B20, A8-B20, A9-B20, A10-B20, A11-B20, A12-B20, A13-B20, A14-B20, A15-B20, A16-B20, A17-B20, A18-B20, A19-B20, A20-B20, A21-B20, A22-B20, A23-B20, A24-B20, A25-B20, A26-B20, A27-B20, A28-B20, A29-B20, A30-B20, A31-B20, A32-B20, A33-B20, A1-B21, A2-B21, A3-B21, A4-B21, A5-B21, A6-B21, A7-B21, A8-B21, A9-B21, A10-B21, A11-B21, A12-B21, A13-B21, A14-B21, A15-B21, A16-B21, A17-B21, A18-B21, A19-B21, A20-B21, A21-B21, A22-B21, A23-B21, A24-B21, A25-B21, A26-B21, A27-B21, A28-B21, A29-B21, A30-B21, A31-B21, A32-B21, A33-B21, A1-B22, A2-B22, A3-B22, A4-B22, A5-B22, A6-B22, A7-B22, A8-B22, A9-B22, A10-B22, A11-B22, A12-B22, A13-B22, A14-B22, A15-B22, A16-B22, A17-B22, A18-B22, A19-B22, A20-B22, A21-B22, A22-B22, A23-B22, A24-B22, A25-B22, A26-B22, A27-B22, A28-B22, A29-B22, A30-B22, A31-B22, A32-B22, A33-B22, A1-B23, A2-B23, A3-B23, A4-B23, A5-B23, A6-B23, A7-B23, A8-B23, A9-B23, A10-B23, A11-B23, A12-B23, A13-B23, A14-B23, A15-B23, A16-B23, A17-B23, A18-B23, A19-B23, A20-B23, A21-B23, A22-B23, A23-B23, A24-B23, A25-B23, A26-B23, A27-B23, A28-B23, A29-B23, A30-B23, A31-B23, A32-B23, A33-B23, A1-B24, A2-B24, A3-B24, A4-B24, A5-B24, A6-B24, A7-B24, A8-B24, A9-B24, A10-B24, A11-B24, A12-B24, A13-B24, A14-B24, A15-B24, A16-B24, A17-B24, A18-B24, A19-B24, A20-B24, A21-B24, A22-B24, A23-B24, A24-B24, A25-B24, A26-B24, A27-B24, A28-B24, A29-B24, A30-B24, A31-B24, A32-B24, A33-B24, A1-B25, A2-B25, A3-B25, A4-B25, A5-B25, A6-B25, A7-B25, A8-B25, A9-B25, A10-B25, A11-B25, A12-B25, A13-B25, A14-B25, A15-B25, A16-B25, A17-B25, A18-B25, A19-B25, A20-B25, A21-B25, A22-B25, A23-B25, A24-B25, A25-B25, A26-B25, A27-B25, A28-B25, A29-B25, A30-B25, A31-B25, A32-B25, A33-B25, A1-B26, A2-B26, A3-B26, A4-B26, A5-B26, A6-B26, A7-B26, A8-B26, A9-B26, A10-B26, A11-B26, A12-B26, A13-B26, A14-B26, A15-B26, A16-B26, A17-B26, A18-B26, A19-B26, A20-B26, A21-B26, A22-B26, A23-B26, A24-B26, A25-B26, A26-B26, A27-B26, A28-B26, A29-B26, A30-B26, A31-B26, A32-B26, A33-B26, A1-B27, A2-B27, A3-B27, A4-B27, A5-B27, A6-B27, A7-B27, A8-B27, A9-B27, A10-B27, A11-B27, A12-B27, A13-B27, A14-B27, A15-B27, A16-B27, A17-B27, A18-B27, A19-B27, A20-B27, A21-B27, A22-B27, A23-B27, A24-B27, A25-B27, A26-B27, A27-B27, A28-B27, A29-B27, A30-B27, A31-B27, A32-B27, A33-B27, A1-B28, A2-B28, A3-B28, A4-B28, A5-B28, A6-B28, A7-B28, A8-B28, A9-B28, A10-B28, A11-B28, A12-B28, A13-B28, A14-B28, A15-B28, A16-B28, A17-B28, A18-B28, A19-B28, A20-B28, A21-B28, A22-B28, A23-B28, A24-B28, A25-B28, A26-B28, A27-B28, A28-B28, A29-B28, A30-B28, A31-B28, A32-B28, A33-B28, A1-B29, A2-B29, A3-B29, A4-B29, A5-B29, A6-B29, A7-B29, A8-B29, A9-B29, A10-B29, A11-B29, A12-B29, A13-B29, A14-B29, A15-B29, A16-B29, A17-B29, A18-B29, A19-B29, A20-B29, A21-B29, A22-B29, A23-B29, A24-B29, A25-B29, A26-B29, A27-B29, A28-B29, A29-B29, A30-B29, A31-B29, A32-B29, A33-B29, A1-B30, A2-B30, A3-B30, A4-B30, A5-B30, A6-B30, A7-B30, A8-B30, A9-B30, A10-B30, A11-B30, A12-B30, A13-B30, A14-B30, A15-B30, A16-B30, A17-B30, A18-B30, A19-B30, A20-B30, A21-B30, A22-B30, A23-B30, A24-B30, A25-B30, A26-B30, A27-B30, A28-B30, A29-B30, A30-B30, A31-B30, A32-B30, A33-B30, A1-B31, A2-B31, A3-B31, A4-B31, A5-B31, A6-B31, A7-B31, A8-B31, A9-B31, A10-B31, A11-B31, A12-B31, A13-B31, A14-B31, A15-B31, A16-B31, A17-B31, A18-B31, A19-B31, A20-B31, A21-B31, A22-B31, A23-B31, A24-B31, A25-B31, A26-B31, A27-B31, A28-B31, A29-B31, A30-B31, A31-B31, A32-B31, A33-B31, A1-B32, A2-B32, A3-B32, A4-B32, A5-B32, A6-B32, A7-B32, A8-B32, A9-B32, A10-B32, A11-B32, A12-B32, A13-B32, A14-B32, A15-B32, A16-B32, A17-B32, A18-B32, A19-B32, A20-B32, A21-B32, A22-B32, A23-B32, A24-B32, A25-B32, A26-B32, A27-B32, A28-B32, A29-B32, A30-B32, A31-B32, A32-B32, A33-B32, A1-B33, A2-B33, A3-B33, A4-B33, A5-B33, A6-B33, A7-B33, A8-B33, A9-B33, A10-B33, A11-B33, A12-B33, A13-B33, A14-B33, A15-B33, A16-B33, A17-B33, A18-B33, A19-B33, A20-B33, A21-B33, A22-B33, A23-B33, A24-B33, A25-B33, A26-B33, A27-B33, A28-B33, A29-B33, A30-B33, A31-B33, A32-B33, A33-B33, A1-B34, A2-B34, A3-B34, A4-B34, A5-B34, A6-B34, A7-B34, A8-B34, A9-B34, A10-B34, A11-B34, A12-B34, A13-B34, A14-B34, A15-B34, A16-B34, A17-B34, A18-B34, A19-B34, A20-B34, A21-B34, A22-B34, A23-B34, A24-B34, A25-B34, A26-B34, A27-B34, A28-B34, A29-B34, A30-B34, A31-B34, A32-B34, A33-B34, A1-B35, A2-B35, A3-B35, A4-B35, A5-B35, A6-B35, A7-B35, A8-B35, A9-B35, A10-B35, A11-B35, A12-B35, A13-B35, A14-B35, A15-B35, A16-B35, A17-B35, A18-B35, A19-B35, A20-B35, A21-B35, A22-B35, A23-B35, A24-B35, A25-B35, A26-B35, A27-B35, A28-B35, A29-B35, A30-B35, A31-B35, A32-B35, A33-B35, A1-B36, A2-B36, A3-B36, A4-B36, A5-B36, A6-B36, A7-B36, A8-B36, A9-B36, A10-B36, A11-B36, A12-B36, A13-B36, A14-B36, A15-B36, A16-B36, A17-B36, A18-B36, A19-B36, A20-B36, A21-B36, A22-B36, A23-B36, A24-B36, A25-B36, A26-B36, A27-B36, A28-B36, A29-B36, A30-B36, A31-B36, A32-B36, A33-B36, A1-B37, A2-B37, A3-B37, A4-B37, A5-B37, A6-B37, A7-B37, A8-B37, A9-B37, A10-B37, A11-B37, A12-B37, A13-B37, A14-B37, A15-B37, A16-B37, A17-B37, A18-B37, A19-B37, A20-B37, A21-B37, A22-B37, A23-B37, A24-B37, A25-B37, A26-B37, A27-B37, A28-B37, A29-B37, A30-B37, A31-B37, A32-B37, A33-B37, A1-B38, A2-B38, A3-B38, A4-B38, A5-B38, A6-B38, A7-B38, A8-B38, A9-B38, A10-B38, A11-B38, A12-B38, A13-B38, A14-B38, A15-B38, A16-B38, A17-B38, A18-B38, A19-B38, A20-B38, A21-B38, A22-B38, A23-B38, A24-B38, A25-B38, A26-B38, A27-B38, A28-B38, A29-B38, A30-B38, A31-B38, A32-B38, A33-B38, A1-B39, A2-B39, A3-B39, A4-B39, A5-B39, A6-B39, A7-B39, A8-B39, A9-B39, A10-B39, A11-B39, A12-B39, A13-B39, A14-B39, A15-B39, A16-B39, A17-B39, A18-B39, A19-B39, A20-B39, A21-B39, A22-B39, A23-B39, A24-B39, A25-B39, A26-B39, A27-B39, A28-B39, A29-B39, A30-B39, A31-B39, A32-B39, and A33-B39. The A moiety is joined to the B moiety to form the ester bond as shown in Formula 3 above.

A pyrethroid prepared according to the methods of the invention can have, for example, a structure selected from:

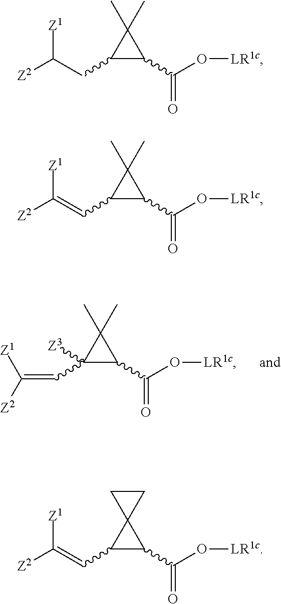

Z¹, Z², and Z³ are independently selected from H, halo, optionally substituted C₁₋₆ alkyl, and optionally substituted C₆₋₁₀ aryl. Z¹ and Z² can also be taken together to form an optionally substituted 5- to 6-membered cycloalkyl or heterocyclyl group.

In some embodiments, the methods of the invention can be used to prepare pyrethroid intermediate compounds that can be converted to the pyrethroid compounds described above. Alkyl esters of cyclopropanecarboxylic acid and cyclopropanecarboxylic acid derivatives can be converted to a variety of pyrethroid compounds via reaction with appropriate alcohols.

Accordingly, some embodiments of the invention provide methods as wherein the cyclopropanation product is a compound according to Formula 2:

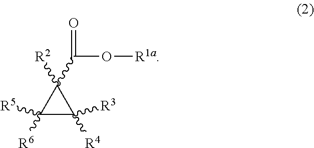

For compounds of Formula 2: R^{1a} is C₁₋₆ alkyl and R² is selected from H and optionally substituted C₆₋₁₀ aryl. In some embodiments, R² is H. In some embodiments, the compound of Formula 2 is selected from:

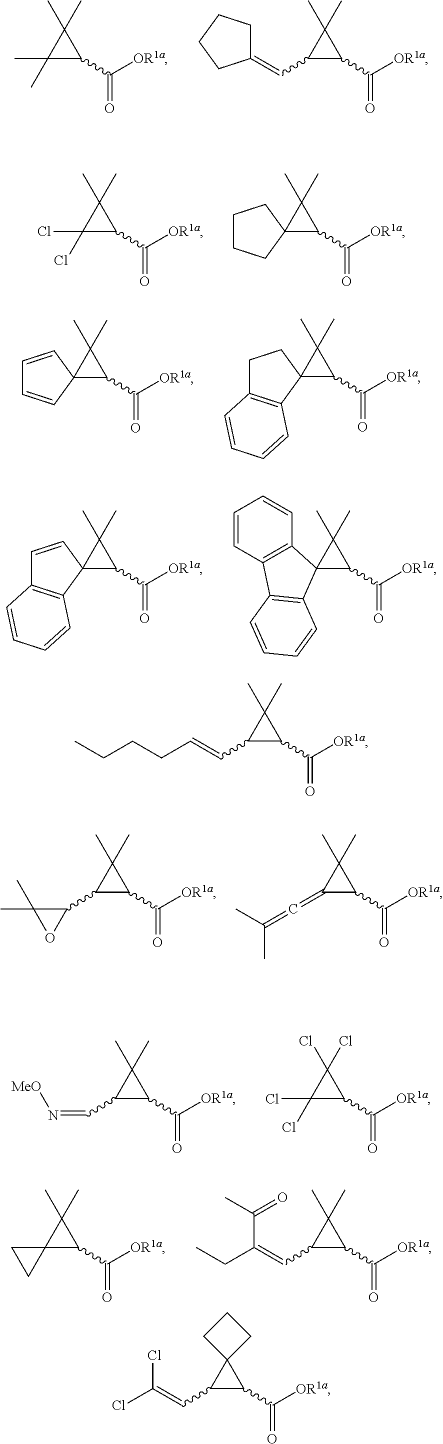

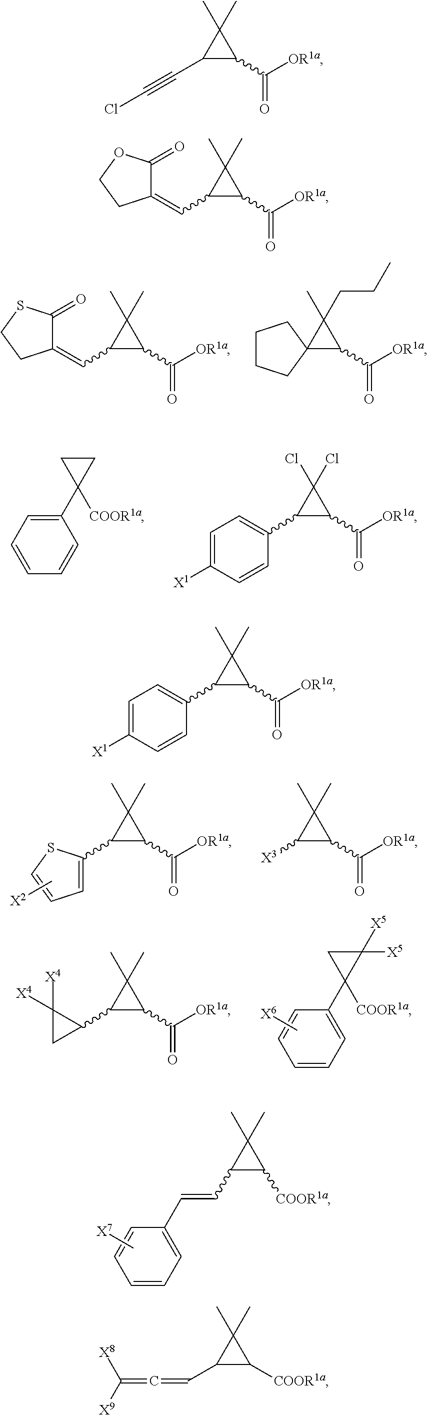

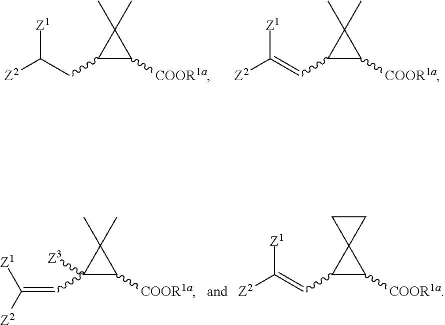

In such embodiments, X¹ is selected from H, optionally substituted C₁₋₆ alkyl, haloC₁₋₆ alkyl, optionally substituted C₁₋₆ alkoxy, optionally substituted C₁₋₆ alkylthio, C₁₋₆ alkylsilyl, halo, and cyano. X² is selected from H, chloro, and methyl. X³ is selected from H, methyl, halo, and CN. Each X⁴ is independently halo. Each X⁵ is independently selected from methyl and halo. X⁶ is selected from halo, optionally substituted C₁₋₆ alkyl, and optionally substituted C₁₋₆ alkoxy. X⁷ is selected from H, methyl, and halo. X⁸ is selected from H, halo, and optionally substituted C₁₋₆ alkyl. X⁹ is selected from H, halo, optionally substituted C₁₋₆ alkyl, C(O)O—(C₁₋₆ alkyl), C(O)—N(C₁₋₆ alkyl)₂, and cyano. Z¹, Z², and Z³ are independently selected from H, halo, optionally substituted C₁₋₆ alkyl, and optionally substituted C₆₋₁₀ aryl. Z¹ and Z² can also be taken together to form an optionally substituted 5- to 6-membered cycloalkyl or heterocyclyl group.

In some embodiments, the methods of the invention include converting the cyclopropanation product according to Formula 2 to a compound according to Formula 3:

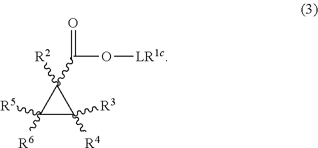

For compounds of Formula 3, L is selected from a bond, —C(R^{L})₂—, and —NR^{L}—C(R^{L})₂—. Each R^{L} is independently selected from H, —CN, and —SO₂. R^{1c} is selected from optionally substituted C₆₋₁₀ aryl, optionally substituted 6- to 10-membered heteroraryl, and optionally substituted 6- to 10-membered heterocyclyl. In some embodiments, L in the compounds of Formula 3 is selected from a bond, —CH₂—, —CH(CN)—, and —N(SO₂)—CH₂.

In some embodiments, the moiety L-R″ in the compounds according to Formula 3 has a structure selected from:

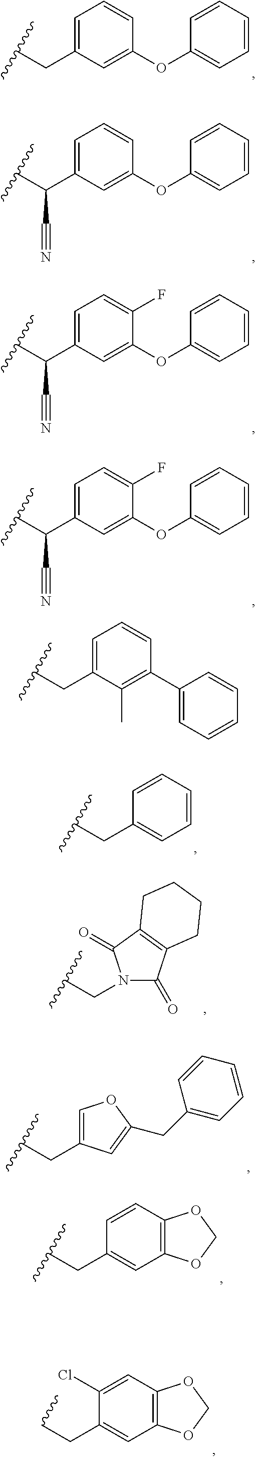

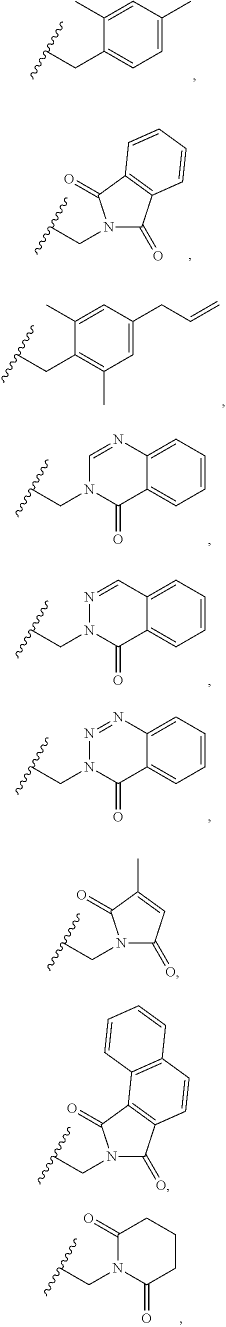

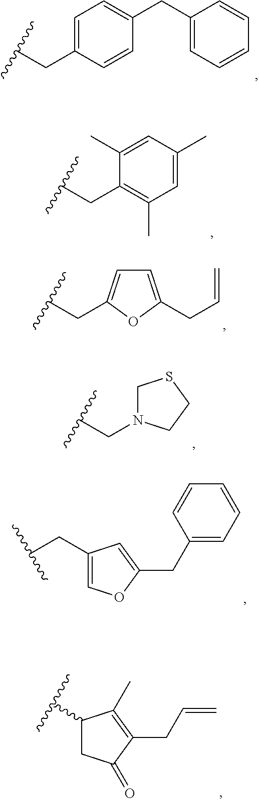

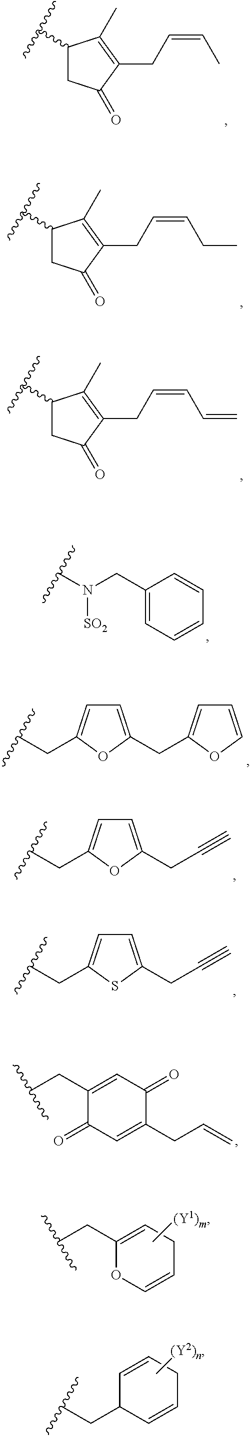

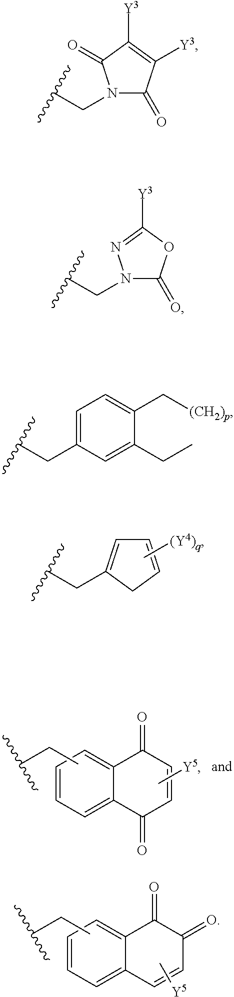

In such embodiments, each Y¹ is independently selected from optionally substituted C₁₋₆ alkyl, optionally substituted C₂₋₆ alkenyl, optionally substituted C₂₋₆ alkynyl, phenyl, and (phenyl)C₁₋₆ alkoxy. Each Y² is independently selected from halo, optionally substituted C₁₋₆ alkyl, optionally substituted C₂₋₆ alkenyl, optionally substituted C₁₋₆ alkoxy, and nitro. Each Y³ is independently optionally substituted C₁₋₆ alkyl. Each Y⁴ is independently selected from optionally substituted C₁₋₆ alkyl, optionally substituted C₂₋₆ alkenyl, optionally substituted C₂₋₆ alkynyl, C₆₋₁₀ aryl-C₁₋₆ alkyl, furfuryl, C₁₋₆ alkoxy, (C₂₋₆ alkenyl)oxy, C₁₋₁₂ acyl, and halo. Y⁵ is selected from optionally substituted C₁₋₆ alkyl, optionally substituted C₁₋₆ alkoxy, and halo. The subscript m is an integer from 1 to 3, the subscript n is an integer from 1 to 5, the subscript p is an integer from 1 to 4, and the subscript q is an integer from 0 to 3. The wavy line at the left of each structure represents the point of connection between the moiety -L-R^{1c} and the rest of the compound according to Formula 3.

In some embodiments, the compound of Formula 3 is selected from:

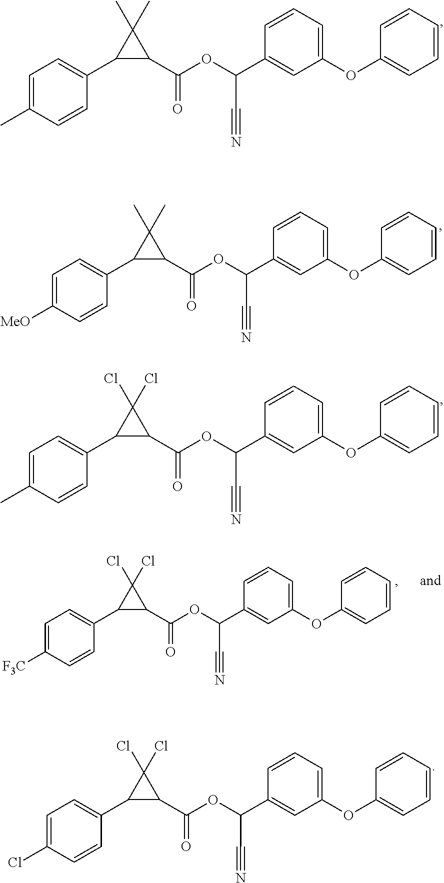 [SMILES: Cc1ccc(C2C(C(=O)OC(C#N)c3cccc(Oc4ccccc4)c3)C2(C)C)cc1 ; COc1ccc(C2C(C(=O)OC(C#N)c3cccc(Oc4ccccc4)c3)C2(C)C)cc1 ; Cc1ccc(C2C(C(=O)OC(C#N)c3cccc(Oc4ccccc4)c3)C2(Cl)Cl)cc1 ; N#CC(OC(=O)C1C(c2ccc(C(F)(F)F)cc2)C1(Cl)Cl)c1cccc(Oc2ccccc2)c1 ; N#CC(OC(=O)C1C(c2ccc(Cl)cc2)C1(Cl)Cl)c1cccc(Oc2ccccc2)c1]

In some embodiments, the compound of Formula 3 is resmethrin.

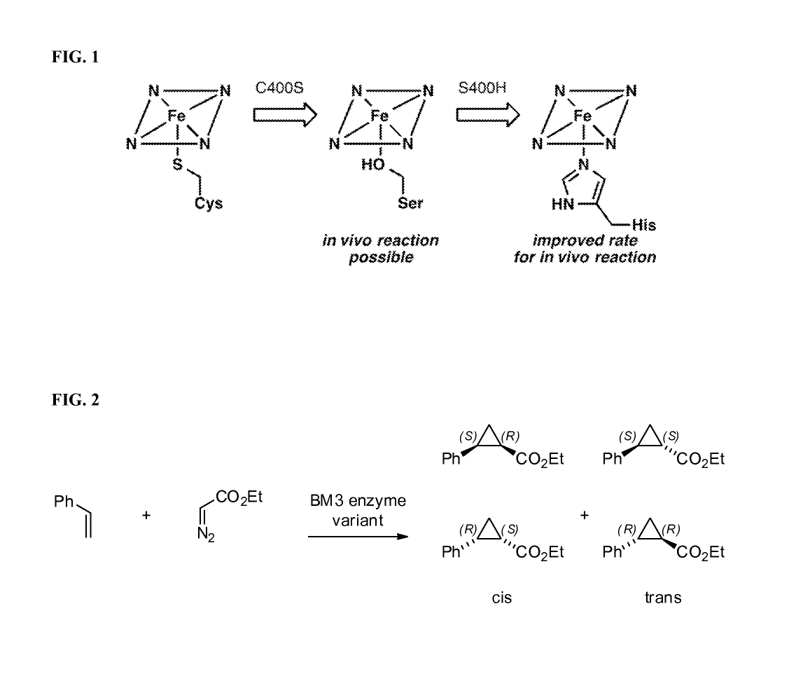

One of skill in the art will appreciate that stereochemical configuration of the cyclopropanation product will be determined in part by the orientation of the diazo reagent with respect to the position of an olefinic substrate such as styrene during the cyclopropanation step. For example, any substituent originating from the olefinic substrate can be positioned on the same side of the cyclopropyl ring as a substituent origination from the diazo reagent. Cyclopropanation products having this arrangement are called “cis” compounds or “Z” compounds. Any substituent originating from the olefinic substrate and any substituent originating from the diazo reagent can also be on opposite sides of the cyclopropyl ring. Cyclopropanation products having this arrangement are called “trans” compounds or “E” compounds. An example of such arrangements is shown in the reaction scheme of FIG. 2.

As shown in FIG. 2, two cis isomers and two trans isomers can arise from the reaction of an olefinic substrate with a diazo reagent. The two cis isomers are enantiomers with respect to one another, in that the structures are non-superimposable mirror images of each other. Similarly, the two trans isomers are enantiomers. One of skill in the art will appreciate that the absolute stereochemistry of a cyclopropranation product—that is, whether a given chiral center exhibits the right-handed “R” configuration or the left-handed “S” configuration-will depend on factors including the structures of the particular olefinic substrate and diazo reagent used in the reaction, as well as the identity of the enzyme. This is also true for the relative stereochemistry—that is, whether a cyclopropanation product exhibits a cis or trans configuration—as well as for the distribution of cyclopropanation product mixtures will also depend on such factors.

In general, cyclopropanation product mixtures have cis:trans ratios ranging from about 1:99 to about 99:1. The cis:trans ratio can be, for example, from about 1:99 to about 1:75, or from about 1:75 to about 1:50, or from about 1:50 to about 1:25, or from about 99:1 to about 75:1, or from about 75:1 to about 50:1, or from about 50:1 to about 25:1. The cis:trans ratio can be from about 1:80 to about 1:20, or from about 1:60 to about 1:40, or from about 80:1 to about 20:1 or from about 60:1 to about 40:1. The cis:trans ratio can be about 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, or about 1:95. The cis:trans ratio can be about 5:1, 10:1, 15:1, 20:1, 25:1, 30:1, 35:1, 40:1, 45:1, 50:1, 55:1, 60:1, 65:1, 70:1, 75:1, 80:1, 85:1, 90:1, or about 95:1.

The distribution of a cyclopropanation product mixture can be assessed in terms of the enantiomeric excess, or “% ee,” of the mixture. The enantiomeric excess refers to the difference in the mole fractions of two enantiomers in a mixture. Taking the reaction scheme in FIG. 2 as a non-limiting example, for instance, the enantiomeric excess of the “E” or trans (R,R) and (S,S) enantiomers can be calculated using the formula: % ee_{E}=[χ_{R,R}−χ_{S,S})/(χ_{R,R}+χ_{S,S})]×100%, wherein χ is the mole fraction for a given enantiomer. The enantiomeric excess of the “Z” or cis enantiomers (% ee_{Z}) can be calculated in the same manner.

In general, cyclopropanantion product mixtures exhibit % ee values ranging from about 1% to about 99%, or from about −1% to about −99%. The closer a given % ee value is to 99% (or −99%), the purer the reaction mixture is. The % ee can be, for example, from about −90% to about 90%, or from about −80% to about 80%, or from about −70% to about 70%, or from about −60% to about 60%, or from about −40% to about 40%, or from about −20% to about 20%. The % ee can be from about 1% to about 99%, or from about 20% to about 80%, or from about 40% to about 60%, or from about 1% to about 25%, or from about 25% to about 50%, or from about 50% to about 75%. The % ee can be from about −1% to about −99%, or from about −20% to about −80%, or from about −40% to about −60%, or from about −1% to about −25%, or from about −25% to about −50%, or from about −50% to about −75%. The % ee can be about −99%, −95%, −90%, −85%, −80%, −75%, −70%, −65%, −60%, −55%, −50%, −45%, −40%, −35%, −30%, −25%, −20%, −15%, −10%, 10%, 15%, 20%, 25%, 30%, 35%, 40%, 45%, 50%, 55%, 60%, 65%, 70%, 75%, 80%, 85%, 90%, or about 95%. Any of these values can be % ee_{E} values or % ee_{Z} values.

Accordingly, some embodiments of the invention provide methods for producing a plurality of cyclopropanation products having a % ee_{Z} of from about −90% to about 90%. In some embodiments, the % ee_{Z} is at least 90%. In some embodiments, the % ee_{Z} is at least −99%. In some embodiments, the % ee_{E} is from about −90% to about 90%. In some embodiments, the % ee_{E} is at least 90%. In some embodiments, the % ee_{E} is at least −99%.

In a related aspect, certain embodiments of the invention provide cyclopropane-containing compounds according to any of Formulas 1, 2, and 3 as described herein. The compounds are prepared using the methods of the invention. In some embodiments, the invention provides a pyrethroid prepared according to the methods of the invention. In some embodiments, the invention provides milnacipran, levomilnacipran, bicifadine, or 1-(3,4-dichlorophenyl)-3-azabicyclo[3.1.0]hexane prepared according to the methods of the invention. In some embodiments, the invention provides any of the compounds illustrated in Table 2, which compounds are prepared according to the methods of the invention. The invention can provide other compounds prepared according to the methods described herein.

C. Reaction Conditions

In a closely related aspect, the invention provides a method for producing a cyclopropanation product. The method includes forming a reaction mixture comprising an olefinic substrate, a carbene precursor, and a cytochrome P450 BM3 enzyme variant under conditions sufficient to produce the cyclopropanation product, wherein the cytochrome P450 BM3 enzyme variant comprises a C400H mutation and one or more mutations selected from V78M, L181V, and L437M relative to the amino acid sequence set forth in SEQ ID NO: 1.

The methods of the invention include forming reaction mixtures that contain a cytochrome P450 BM3 enzyme variant described herein. The cytochrome P450 BM3 enzyme variant can be, for example, purified prior to addition to a reaction mixture or secreted by a cell present in the reaction mixture. The reaction mixture can contain a cell lysate including the enzyme variant, as well as other proteins and other cellular materials. Alternatively, a cytochrome P450 BM3 enzyme variant can catalyze the reaction within a cell expressing the enzyme variant. Any suitable amount of the cytochrome P450 BM3 enzyme variant can be used in the methods of the invention. In general, cyclopropanation reaction mixtures contain from about 0.01 mol % to about 10 mol % cytochrome P450 BM3 enzyme variant with respect to the diazo reagent and/or olefinic substrate. The reaction mixtures can contain, for example, from about 0.01 mol % to about 0.1 mol % cytochrome P450 BM3 enzyme variant, or from about 0.1 mol % to about 1 mol % cytochrome P450 BM3 enzyme variant, or from about 1 mol % to about 10 mol % cytochrome P450 BM3 enzyme variant. The reaction mixtures can contain from about 0.05 mol % to about 5 mol % cytochrome P450 BM3 enzyme variant, or from about 0.05 mol % to about 0.5 mol % cytochrome P450 BM3 enzyme variant. The reaction mixtures can contain about 0.1, 0.2, 0.3, 0.4, 0.5, 0.6, 0.7, 0.8, 0.9, or about 1 mol % cytochrome P450 BM3 enzyme variant.

Any olefinic substrate or diazo reagent as described herein can be used in the methods of the invention. The concentration of olefinic substrate and diazo reagent are typically in the range of from about 100 μM to about 1 M. The concentration can be, for example, from about 100 μM to about 1 mM, or about from 1 mM to about 100 mM, or from about 100 mM to about 500 mM, or from about 500 mM to 1 M. The concentration can be from about 500 μM to about 500 mM, 500 μM to about 50 mM, or from about 1 mM to about 50 mM, or from about 15 mM to about 45 mM, or from about 15 mM to about 30 mM. The concentration of olefinic substrate or diazo reagent can be, for example, about 100, 200, 300, 400, 500, 600, 700, 800, or 900 μM. The concentration of olefinic substrate or diazo reagent can be about 1, 5, 10, 15, 20, 25, 30, 35, 40, 45, 50, 55, 60, 65, 70, 75, 80, 85, 90, 95, 100, 150, 200, 250, 300, 350, 400, 450, or 500 mM.

Reaction mixtures can contain additional reagents. As non-limiting examples, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, isopropanol, glycerol, tetrahydrofuran, acetone, acetonitrile, and acetic acid), salts (e.g., NaCl, KCl, CaCl₂, and salts of Mn²⁺ and Mg²⁺), denaturants (e.g., urea and guandinium hydrochloride), detergents (e.g., sodium dodecylsulfate and Triton-X 100), chelators (e.g., ethylene glycol-bis(2-aminoethylether)-N,N,N′,N′-tetraacetic acid (EGTA), 2-({2-[Bis(carboxymethyl)amino]ethyl}(carboxymethyl)amino)acetic acid (EDTA), and 1,2-bis(o-aminophenoxy)ethane-N,N,N,N-tetraacetic acid (BAPTA)), sugars (e.g., glucose, sucrose, and the like), and reducing agents (e.g., sodium dithionite, NADPH, dithiothreitol (DTT), β-mercaptoethanol (BME), and tris(2-carboxyethyl)phosphine (TCEP)). Buffers, cosolvents, salts, denaturants, detergents, chelators, sugars, and reducing agents can be used at any suitable concentration, which can be readily determined by one of skill in the art. In general, buffers, cosolvents, salts, denaturants, detergents, chelators, sugars, and reducing agents, if present, are included in reaction mixtures at concentrations ranging from about 1 μM to about 1 M. For example, a buffer, a cosolvent, a salt, a denaturant, a detergent, a chelator, a sugar, or a reducing agent can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In some embodiments, a reducing agent is used in a sub-stoichiometric amount with respect to the olefin substrate and the diazo reagent. Cosolvents, in particular, can be included in the reaction mixtures in amounts ranging from about 1% v/v to about 75% v/v, or higher. A cosolvent can be included in the reaction mixture, for example, in an amount of about 5, 10, 20, 30, 40, or 50% (v/v). Accordingly, some embodiments of the invention provide a reaction mixture as described above, wherein the reaction mixture further comprises a reducing agent.

Reactions are conducted under conditions sufficient to catalyze the formation of a cyclopropanation product. The reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 6 to about 10. The reactions can be conducted, for example, at a pH of from about 6.5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Reactions can be conducted under aerobic conditions or anaerobic conditions. Reactions can be conducted under an inert atmosphere, such as a nitrogen atmosphere or argon atmosphere. In some embodiments, a solvent is added to the reaction mixture. In some embodiments, the solvent forms a second phase, and the cyclopropanation occurs in the aqueous phase. In some embodiments, the cytochrome P450 BM3 enzyme variant is located in the aqueous layer whereas the substrates and/or products occur in an organic layer. Other reaction conditions may be employed in the methods of the invention, depending on the identity of a particular cytochrome P450 BM3 enzyme variant, olefinic substrate, or diazo reagent.

Reactions can be conducted in vivo with intact cells expressing a cytochrome P450 BM3 enzyme variant of the invention. The in vivo reactions can be conducted with any of the host cells used for expression of the cytochrome P450 BM3 enzyme variant, as described herein. Accordingly, some embodiments of the invention provide methods as described above wherein the cytochrome P450 BM3 enzyme variant is localized within a whole cell and the cyclopropanation product is produced in vivo. A suspension of cells can be formed in a suitable medium supplemented with nutrients (such as mineral micronutrients, glucose and other fuel sources, and the like). Cyclopropanation yields from reactions in vivo can be controlled, in part, by controlling the cell density in the reaction mixtures. Cellular suspensions exhibiting optical densities ranging from about 0.1 to about 50 at 600 nm can be used for cyclopropanation reactions. Other densities can be useful, depending on the cell type, specific cytochrome P450 BM3 enzyme variant, or other factors.

The methods of the invention can be assessed in terms of the diastereoselectivity and/or enantioselectivity of cyclopropanation reaction, that is, the extent to which the reaction produces a particular isomer, whether a diastereomer or enantiomer. A perfectly selective reaction produces a single isomer, such that the isomer constitutes 100% of the product. As another non-limiting example, a reaction producing a particular enantiomer constituting 90% of the total product can be said to be 90% enantioselective. A reaction producing a particular diastereomer constituting 30% of the total product, meanwhile, can be said to be 30% diastereoselective.

In general, the methods of the invention include reactions that are from about 1% to about 99% diastereoselective. The reactions are from about 1% to about 99% enantioselective. The reaction can be, for example, from about 10% to about 90% diastereoselective, or from about 20% to about 80% diastereoselective, or from about 40% to about 60% diastereoselective, or from about 1% to about 25% diastereoselective, or from about 25% to about 50% diastereoselective, or from about 50% to about 75% diastereoselective. The reaction can be about 10%, 15%, 20%, 25%, 30%, 35%, 40%, 45%, 50%, 55%, 60%, 65%, 70%, 75%, 80%, 85%, 90%, or about 95% diastereoselective. The reaction can be from about 10% to about 90% enantioselective, from about 20% to about 80% enantioselective, or from about 40% to about 60% enantioselective, or from about 1% to about 25% enantioselective, or from about 25% to about 50% enantioselective, or from about 50% to about 75% enantioselective. The reaction can be about 10%, 15%, 20%, 25%, 30%, 35%, 40%, 45%, 50%, 55%, 60%, 65%, 70%, 75%, 80%, 85%, 90%, or about 95% enantioselective. Accordingly some embodiments of the invention provide methods wherein the reaction is at least 30% to at least 90% diastereoselective. In some embodiments, the reaction is at least 30% to at least 90% enantioselective.

### IV. Examples

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Cytochrome P450-Catalyzed Cyclopropanation of Electron-Deficient Acrylamides in Whole Cells

Small Scale Whole Cell Reactions.

E. coli (BL21) cells coding for appropriate enzyme variant were grown from glycerol stock overnight (37° C., 250 rpm) in 5 ml TBₐₘₚ. The pre-culture was used to inoculate 45 mL of Hyperbroth medium (1 L Hyperbroth prepared from powder from AthenaES©, 0.1 mg mL⁻¹ ampicillin) in a 125 mL Erlenmeyer flask and this culture was incubated at 37° C., 200 rpm for approximately 3 h. At OD₆₀₀=1.8, the cultures were cooled to 22° C. and the shaking was reduced to 140 rpm before inducing with IPTG (0.25 mM) and δ-aminolevulinic acid (0.50 mM). Cultures were harvested after 20 h and resuspended in nitrogen-free M9-N medium (1 L: 31 g Na₂HPO₄, 15 g KH₂PO₄, 2.5 g NaCl, 0.24 g MgSO₄, 0.010 g CaCl₂) until the indicated OD₆₀₀ (usually OD₆₀₀=60) is obtained. Aliquots of the cell suspension were used for determination of the enzyme expression level (2-3 mL) after lysis.

Anaerobic Conditions.

E. coli cells (OD₆₀₀=60) were transferred to a crimped 6 mL vial and made anaerobic by degassing with argon for 5-10 min. In parallel, glucose (50 μL, 250 mM) was added to 2 mL crimp vials that are sealed. The headspaces of these vials were purged with argon for 5-10 min. If multiple reactions were being carried out in parallel, a maximum of 8 vials were connected via cannulae and degassed in series. Cells (425 μL) were transferred to each vial via syringe and the olefin substrate was added (12.5 μL of a 800 mM solution of styrene in EtOH or a 400 mM solution of acrylamide 1 in EtOH), followed by EDA (12.5 μL of a 350 mM or 400 mM solution in EtOH). The reactions were shaken on a table-top shake plate at room temperature for 5 h. The reactions were quenched by addition of 25 μL of 3 M HCl, followed by 20 μL of the internal standard (20 mM 2-phenylethanol solution in cyclohexane) and 1 mL cyclohexane. The mixture was transferred to a 2 mL Eppendorf tube, vortexed and then centrifuged (10,000× rcf, 30 s). The organic layer was removed and analyzed by GC to determine yield and chiral SFC to determine enantioselectivity.

Aerobic Conditions.

Cell suspension was used without sparging with argon. Cells (425 μL, OD₆₀₀=60) and glucose (50 μL, 250 mM) were combined in an unsealed 2 mL glass vial. The olefin substrate was added (12.5 μL, 400 mM in EtOH), followed by EDA (12.5 μL, 400 mM in EtOH). The vial was covered with foil then shaken at 35 rpm for 5 h. The reactions were quenched by addition of 25 μL of 3 M HCl, followed by 20 μL of the internal standard (20 mM 2-phenylethanol solution in cyclohexane) and 1 mL cyclohexane. The mixture was transferred to a 2 mL Eppendorf tube, vortexed and then centrifuged (10,000x rcf, 30 s). The organic layer was removed and analyzed by GC to determine yield and chiral SFC to determine enantioselectivity.

Analysis of Crude Reaction Mixtures.

GC analysis of product was performed using J&W HP-5 column (30 m×0.32 mm, 0.25 μM film) with the method 90° C. hold 2 min, 90-110 at 6° C./min, 110-190 at 40° C./min, 190-300 at 20° C./min, 300° C. hold 1 min, 12.8 min total): internal standard (3.55 min), retention times for the cis and trans products are listed in the characterization section below. Analytical SFC of product was performed on either Chiralpak AS column or OD column, eluting with iPrOH at 2.5 mL/min and detecting at 210 nm. Semi-preparative HPLC for all products was performed on 9.4 mm×250 mm, 5 μm Agilent XDB-C18 column, detection at 210 nm, flow rate 3.0 mL/min, H₂O/MeCN, gradient: 0 min 10% MeCN, 30 min 70% MeCN, hold 5 min, 40 min 95% MeCN

Calibration of Cyclopropanation Products.

Yields of cyclopropanation products were determined using calibration curves made with independently synthesized standards. Stock solutions of product were made at 120 or 160 mM in DMSO. To 4 samples containing cells at OD₆₀₀=60, product was added from either of the stock solutions such that a final concentration of 1.5-6.0 or 2.0-8.0 mM product was obtained. Additional DMSO was added such that the total volume of organics added to each tube was 25 pt. Next, 20 μL of a 20 mM stock solution of internal standard in cyclohexane was added to each Eppendorf tube, followed by 1 mL of cyclohexane. The Eppendorf tubes were vortexed and centrifuged (13,000×rcm, 30 seconds). The organic layer was then analyzed by GC using J&W HP-5 column (30 m x 0.32 mm, 0.25 μM film: 90° C. hold 2 min, 90-110 at 6° C./min, 110-190 at 40° C./min, 190-300 at 20° C./min, 300° C. hold 1 min, 12.8 min total). The ratio of the areas under the internal standard and product peaks was plotted against the concentration for each solution (1.5 to 6.0 mM or 2.0 to 8.0 mM).

Preparative Scale Whole Cell Aerobic Reactions.

For characterization purposes, the aerobic reactions were scaled up as follows: Cells (8.5 mL, OD₆₀₀=60) and glucose (1.0 mL, 250 mM) were combined in an unsealed scintillation vial. The olefin substrate was added (0.25 mL, 400 mM in EtOH), followed by EDA (0.25 mM, 400 mM in EtOH). The vial was capped and then shaken at 35 rpm for 5 h. The reactions were quenched by addition of 0.25 mL of 3 M HCl, poured into a Falcon tube, extracted with 1:1 EtOAc:hexanes (7.5 mL), and centrifuged (5,000 rpm, 5 min). The organics were collected, and this extraction sequence was repeated once. The organics were combined, dried with Na₂SO₄, and concentrated in vacuo. The crude product was purified via semi-preparative HPLC, with the exception of 10, which was purified by silica gel chromatography (1;1 Hexanes:DCM to 100% DCM).

### Example 2

### Cyclopropanation of Acrylamides with Varying Nitrogen Substituents

A library of acrylamides was synthesized via direct amidation reaction of the corresponding acid chloride, as well as condensation reaction of the appropriate diethyl carboxamide precursors with paraformaldehyde. In certain cases, the substituent on the amide moiety was varied by conducting Schotten-Baumann reactions on atropic acid with the appropriate amines (Scheme 4A). In parallel, a range of phenylacetic acid derivatives was converted to the corresponding diethyl carboxamide, followed by condensation with paraformaldehyde to arrive at the appropriate acrylamides (Scheme 4B). Variation on both the amide and the aryl moieties provided for the examination of the steric and electronic restriction the enzyme scaffold places on the cyclopropanation reaction.

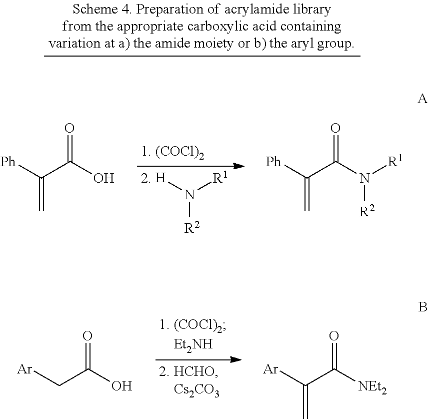

When a variety of small- to medium-sized acrylamides were combined with EDA in the presence of Escherichia coli cells expressing BM3-Hstar, formation of the desired cyclopropanes in more than 90% yield with excellent diastereoselectivity and enantioselectivity was observed (Table 3). The yields and diastereoselectivity obtained when using BM3-Hstar with an acrylamide substrate exceeded yields and selectivity obtained when using a number of other P450-BM3 enzyme variants. See, e.g., U.S. Pat. No. 8,993,262. Notably, Weinreb amide 6b, a valuable intermediate for further transformations (Balasubramaniam, et al., Synthesis, 2008, 23, 3707), could be synthesized in excellent yield and selectivity. Unsymmetrical amides such as 5f could also be cyclopropanated in good yields. Previous biological studies with a racemic sample of tetrahydroquinolinyl analog of milnacipran have shown that it is very active and highly selective for inhibition of epinephrine and serotonin monoamine transporters (Chen et al., Bioorg. Med. Chem. Lett., 2008, 18, 1346). A precursor to this potent molecule, 6g, can be prepared in 50% yield and good selectivity with BM3-Hstar. While the yield of this reaction is lower than what was observed for smaller amide substrates, the method of the invention provides a facile route for rapidly accessing enantioenriched 6g.

TABLE 3
Scope of acrylamides with variation on the amide moiety.^{a,b,c}

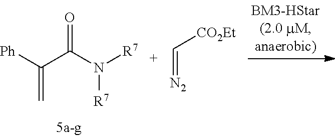

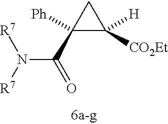

| Product | 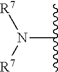 | yield | TTN | dr | ee |
|---|---|---|---|---|---|
| 6a | 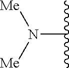 [SMILES: *N(C)C] | 98% | 4900 | 99:1 | 98% |
| 6b | 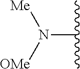 [SMILES: *N(C)OC] | 99% | 5000 | 99:1 | 97% |
| 6c | 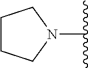 [SMILES: *N1CCCC1] | 98% | 4900 | 98:2 | 91% |
| 6d | 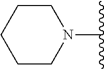 [SMILES: *N1CCCCC1] | 97% | 4900 | 96:4 | 88% |
| 6e | 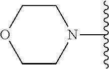 [SMILES: *N1CCOCC1] | 93% | 4700 | 99:1 | 94% |
| 6f | 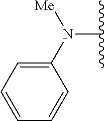 [SMILES: *N(C)c1ccccc1] | 75% | 3800 | 98:2 | 89% |
| 6g | 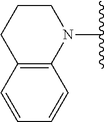 [SMILES: *N1CCCc2ccccc21] | 50% | 2500 | 93:7 | 71% |

^{a}Reactions were carried out with whole cells expressing BM3-HStar (2.0 μM), glucose (250 mM, 50 μM), acrylamide (10 μM), EDA (10 μM) in M9-N buffer (500 μL) at room temperature.
^{b}Yields and d.r. were determined by gas chromatography calibrated for the appropriate products. Enantioselectivity was determined by chiral super-critical fluid chromatography (SFC).
^{c}Relative and absolute configurations were assigned based on analogy to 2.

### Example 3

### Cyclopropanation of Aromatic Acrylamides with Varying Aryl Substituents

Moving to the aryl group of the acrylamide, both sterically- and electronically-demanding aryl substituents were found to be well-tolerated (Table 4). Acrylamides containing electron-rich aryl groups (7a-c) provided the corresponding cyclopropane products in good to high yield and great stereoselectivity and even substrates containing p-Cl or p-CF₃ electron withdrawing substituents (7d and 7e, respectively) were readily cyclopropanated with BM3-Hstar, albeit with lower yields. Additionally, increasing the size of the aryl group to naphthyl did not diminish the yield of the reaction.

BM3-Hstar is surprisingly insensitive to the size and shape of the acrylamide, considering that substrates like 5a and 5g differ by seven carbons and even those with rigid substituents like naphthyl (7f) react readily within the protein. Interestingly, the diastereo- and enantioselectivity of the cyclopropanation remained consistent for all of the substrates examined, suggesting that the enzyme facilitates the approach of the olefin to the putative iron carbenoid generated at the P450-heme prosthetic group in the same orientation for all of the substrates examined.

TABLE 4
Scope of acrylamides with variation on the aryl ring.^{a,b,c}

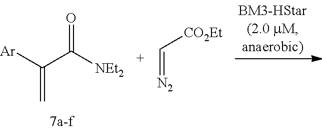

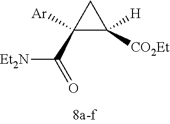

| Product | 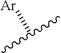 | yield | TTN | dr | ee |
|---|---|---|---|---|---|
| 8a | 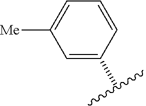 | 80% | 4000 | 97:3 | 87% |
| 8b | 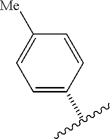 | 76% | 3800 | 93:7 | 66% |
| 8c | 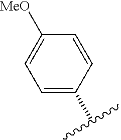 | 82% | 4100 | 96:4 | 88% |
| 8d | 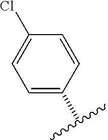 | 83% | 4200 | 91:9 | 59% |
| 8e | 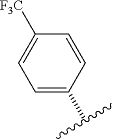 | 77% | 3900 | 94:6 | 58% |
| 8f | 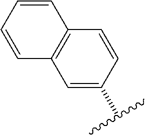 | 80% | 4000 | 96:4 | 82% |

^{a}Reactions were carried out with whole cells expressing BM3-HStar (2.0 μM), glucose (250 mM, 50 μM), acrylamide (10 μM), EDA (10 μM) in M9-N buffer (500 μL) at room temperature.
^{b}Yields and d.r. were determined by GC calibrated for the appropriate products. Enantioselectivity was determined by chiral SFC.
^{c}Relative and absolute configurations were assigned based on analogy to 2.

Changing the acrylamide to the analogous acrylate (Scheme 5), despite having little effect on the yield, led to diminished diastereoselectivity and enantioselectivity of the reaction. This result suggests that the amide group is important for stereocontrol in cyclopropanation with BM3-Hstar and that our evolved protein may be tuned for this particular functional group.

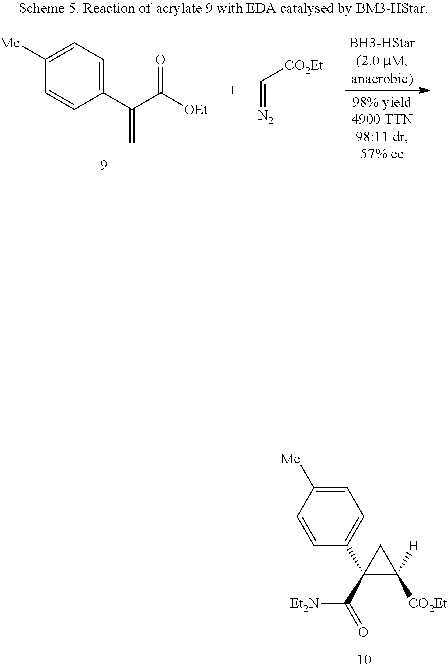

### Example 4

### Aerobic and Anaerobic Cyclopropanation Reactions

Without wishing to be bound by any particular theory, it is believed that the much improved rate of cyclopropanation of acrylamide 1 catalyzed by BM3-HStar can out-compete inhibition by molecular oxygen, thereby allowing the cyclopropanation reaction to be performed under aerobic conditions. To test the generality of this behavior, cyclopropanation of 5a-g, 7a-f, and 9 was performed under ambient atmosphere without degassed buffer or glucose. A comparison of the reaction for each substrate under aerobic versus anaerobic conditions is shown in Table 5. Reactions with substrates 5a-5g showed minimal loss in yield when conducted under aerobic conditions. However, the effect of oxygen inhibition became more appreciable when the substituents on the aryl ring were varied. In particular, electron-withdrawing substituents and the naphthyl group gave the most significant drop in yield when the reaction was run under aerobic conditions. Presumably, the more electron-deficient nature of the olefin and the increase in steric bulk (in the case of naphthyl substrate 7f) led to a slower rate of cyclopropanation and as a result, inhibition by atmospheric oxygen is competitive.

TABLE 5
Comparison of anaerobic and aerobic reactions with BM3-HStar.

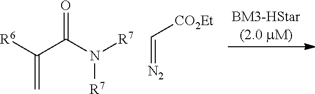

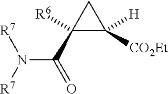

| | | | Aerobic |
|---|---|---|---|
| | Anaerobic | Aerobic | yield/anaerobic |
| Product | Yield (%) | Yield (%) | yield |
| 6a | 98 | 98 | 1.0 |
| 6b | 99 | 98 | 0.99 |
| 6c | 98 | 98 | 1.0 |
| 6d | 97 | 95 | 0.98 |
| 6e | 93 | 92 | 0.99 |
| 6f | 75 | 75 | 1.0 |
| 6g | 50 | 45 | 0.90 |
| 8a | 80 | 71 | 0.89 |
| 8b | 76 | 68 | 0.89 |
| 8c | 82 | 72 | 0.88 |
| 8d | 83 | 61 | 0.73 |
| 8e | 77 | 62 | 0.81 |
| 8f | 80 | 54 | 0.68 |
| 10 | 98 | 95 | 0.97 |

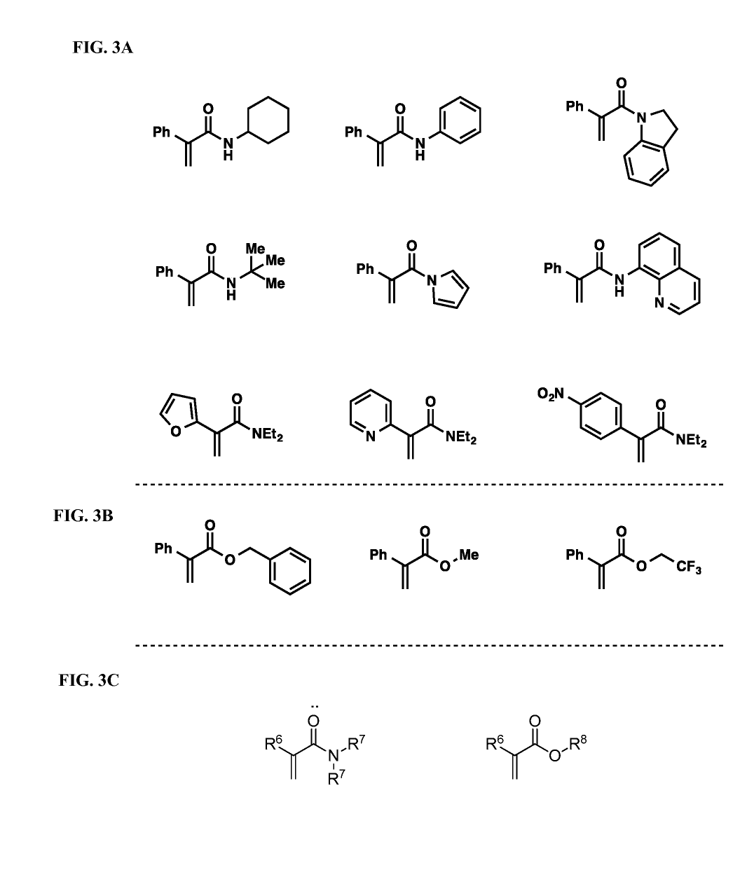

Based on these results, this reaction is widely tolerant to a great range of substitutions on both the amide and the aryl moieties, including secondary and tertiary amides, alkyl and aryl esters and heteroaryl rings (FIG. 3). In addition, simultaneous modifications of both amide and aryl moieties should still be tolerated in the reaction.

### Example 5

### Characterization Data for Cyclopropanes

Compound 6a.

¹H NMR (400 MHz, CDCl₃): δ 7.36-7.20 (m, 5H), 4.19 (ddt, J=7.2, 5.4, 2.6 Hz, 2H), 2.94 (s, 3H), 2.90 (s, 3H), 2.43 (dd, J=8.3, 6.3, 1H), 2.14 (dd, J=6.4, 4.9 Hz, 1H), 1.51 (dd, J=8.4, 5.1 Hz, 1H), 1.28 (td, J=7.2, 1.8 Hz, 3H). ¹³C NMR (CDCl₃, 126 MHz): δ 170.9, 168.5, 138.8, 129.0, 127.4, 126.3, 61.1, 38.8, 37.3, 35.8, 28.9, 21.8, 14.4. HRMS (m/z): calcd for C₁₅H₁₉O₃N, [M+H]⁺, 262.1443. found, 262.1446. GC: Using method described above, t_{R} (min): cis=9.25, trans=9.42. HPLC: Using method described above, t_{R} (min)=21.7. SFC: AS column, 4% IPA, 2.5 mL/min: λ=210 nm, t_{R} (min): major=6.45, minor=7.53.

Compound 6b.

¹H NMR (500 MHz, CDCl₃): δ 7.36-7.20 (m, 5H), 4.39-3.98 (m, 2H), 3.23 (s, 2H), 3.11 (s, 3H), 2.51 (dd, J=8.6, 6.3 Hz, 1H), 2.09 (t, J=5.6 Hz, 1H), 1.58 (d, J=4.4 Hz, 1H), 1.28 (dd, J=7.8, 6.5 Hz, 3H). ¹³C NMR (CDCl₃, 126 MHz): δ 171.4, 138.8, 128.7, 127.8, 127.5, 110.2, 61.1, 60.8, 38.9, 33.6, 26.4, 20.8, 14.3. HRMS (m/z): calcd for C₁₅H₁₉O₄N, [M+H]⁺, 278.1392. found, 278.1398. GC: Using method described above, t_{R} (min): cis=9.22, trans=9.40. HPLC: Using method described above, t_{R}=23.9 min. SFC: OD column, 10% IPA, 2.5 mL/min: λ=210 nm, t_{R} (min): major=3.88, minor=4.40.

Compound 6c.

¹H NMR (500 MHz, CDCl₃): δ 7.36-7.20 (m, 5H), 4.19 (qd, J=7.1, 1.9 Hz, 2H), 3.54-3.43 (m, 2H), 3.39-3.28 (m, 1H), 3.29-3.16 (m, 1H), 2.41 (dd, J=8.4, 6.2 Hz, 1H), 2.18 (dd, J=6.2, 4.9 Hz, 1H), 1.86-1.68 (m, 4H), 1.49 (dd, J=8.4, 4.9 Hz, 1H), 1.29 (t, J=7.1 Hz, 3H). ¹³C NMR (CDCl₃, 126 MHz): δ 171.0, 166.8, 138.6, 128.9, 127.4, 126.7, 61.1, 46.6, 46.4, 40.1, 28.3, 26.2, 24.2, 21.2, 14.4. HRMS (m/z): calcd for C₁₇H₂₁O₃N, [M+H]⁺, 288.1600. found, 288.1591. GC: Using method described above, t_{R} (min): cis=10.51, trans=10.60. HPLC: Using method described above, t_{R}=23.5 min. SFC: AS column, 10% IPA, 2.5 mL/min: λ=210 nm, t_{R} (min): major=5.12, minor=6.54.

Compound 6d.

¹H NMR (500 MHz, CDCl₃): δ 7.36-7.20 (m, 5H), 3.83-3.66 (m, 1H), 3.51 (ddd, J=13.3, 6.7, 3.8 Hz, 1H), 3.45-3.32 (m, 1H), 3.25 (ddd, J=13.3, 8.2, 3.6 Hz, 1H), 2.46 (dd, J=8.4, 6.2 Hz, 1H), 2.16 (dd, J=6.2, 4.9 Hz, 1H), 1.59-1.45 (m, 4H) 1.50 (dd, J=8.3, 5.0 Hz, 1H), 1.29 (t, J=7.1 Hz, 3H), 1.26-1.09 (m, 2H). ¹³C NMR (CDCl₃, 126 MHz): δ 170.9, 166.9, 139.1, 128.9, 128.9, 127.3, 126.4, 61.1, 46.7, 43.3, 38.8, 28.5, 25.7, 25.6, 25.5, 24.6, 21.7, 14.4. HRMS (m/z): calcd for C₁₈H₂₃O₃N, [M+H]⁺, 302.1756. found, 302.1770. GC: Using method described above, t_{R} (min): cis=10.68, trans=10.81. HPLC: Using method described above, t_{R}=27.4 min. SFC: AS column, 10% IPA, 2.5 mL/min: λ=210 nm, t_{R} (min): major=4.06, minor=4.36.

Compound 6e.

¹H NMR (500 MHz, CDCl₃): δ 7.36-7.20 (m, 5H), 4.45-4.05 (m, 2H), 3.71-3.65 (m, 1H), 3.64-3.55 (m, 3H), 3.51-3.43 (m, 1H), 3.42-3.20 (m, 3H), 2.49 (dd, J=8.4, 6.2 Hz, 1H), 2.16 (dd, J=6.2, 4.9 Hz, 1H), 1.50 (dd, J=8.5, 4.9 Hz, 1H), 1.30 (t, J=7.1 Hz, 3H). ¹³C NMR (CDCl₃, 126 MHz): δ 170.7, 167.4, 138.5, 129.1, 129.1, 127.6, 126.2, 66.8, 66.3, 61.3, 46.3, 42.7, 38.4, 28.3, 21.7, 14.4. HRMS (m/z): calcd for C₁₇H₂₁O₄N, [M+H]⁺, 304.1549. found, 304.1538. GC: Using method described above, t_{R} (min): cis=10.60, trans=10.77. HPLC: Using method described above, t_{R} (min)=21.0. SFC: AS column, 5% IPA, 2.5 mL/min: λ=210 nm, t_{R} (min): major=5.98, minor=6.47.

Compound 6f.

¹H NMR (500 MHz, CDCl₃): δ 7.40-7.18 (m, 6H), 7.14-7.09 (m, 2H), 6.94-6.82 (m, 2H), 4.23 (q, J=7.2 Hz, 2H), 3.27 (s, 3H), 1.97 (t, J=7.3 Hz, 1H), 1.73 (t, J=5.9 Hz, 1H), 1.37-1.25 (m, 4H). ¹³C NMR (CDCl₃, 126 MHz): δ 171.5, 168.2, 143.7, 139.7, 129.1, 128.5, 127.9, 127.5, 127.2, 61.1, 39.8, 38.7, 30.1, 21.2, 14.4. HRMS (m/z): calcd for C₂₀H₂₁O₃N, [M+H]⁺, 324.1600. found, 324.1596. GC: Using method described above, t_{R} (min): cis=10.94, trans=11.11. HPLC: Using method described above, t_{R} (min)=30.8. SFC: OD column, 10% IPA, 2.5 mL/min: λ=210 nm, t_{R} (min): major=6.27, minor=7.09.

Compound 6g.

¹H NMR (500 MHz, C₆D₆, 25° C.): δ 8.49 (brs, 1H), 7.59-6.70 (m, 8H), 4.14-3.89 (m, 2H), 3.20 (brs, 1H), 2.70-1.83 (brm, 3H), 2.24 (dt, J=15.8, 6.6 Hz, 1H), 1.41-1.06 (brm, 1H), 1.24 (dd, J=8.2, 5.4 Hz, 1H), 0.97 (t, J=7.1 Hz, 3H). ¹H NMR (500 MHz, C₆D₆, 65° C.): δ 7.68 (brs, 1H), 7.36 (d, J=7.7 Hz, 2H), 7.12-6.81 (m, 6H), 4.15-3.92 (m, 3H), 3.29 (dt, J=12.6, 5.8 Hz, 1H), 2.47 (dd, J=15.2, 7.6 Hz, 1H), 2.31 (dt, J=15.8, 6.6 Hz, 1H), 1.95 (brs, 1H), 1.67 (brs, 1H), 1.37-1.31 (m, 1H), 1.29 (dd, J=8.2, 5.4 Hz, 1H), 1.04 (t, J=7.1, 3H). ¹³C NMR (C₆D₆, 126 MHz): δ 170.8, 167.7, 146.9, 139.9, 129.0, 128.6, 127.3, 126.9, 126.0, 125.4, 124.9, 61.0, 44.4, 39.7, 26.9, 23.8, 21.6, 14.3. HRMS (m/z): calcd for C₂₂H₂₃O₃N, [M+H]⁺, 350.1756. found, 350.1760. GC: Using method described above, t_{R} (min): cis=12.30, trans=12.38. HPLC: Using method described above, t_{R} (min)=35.6. SFC: OD column, 10% IPA, 2.5 mL/min: λ=210 nm, t_{R} (min): major=10.17, minor=11.21.

Compound 8a.

¹H NMR (500 MHz, CDCl₃): δ 7.20 (t, J=7.6 Hz, 1H), 7.14 (td, J=1.6, 0.7 Hz, 1H), 7.07 (dddt, J=12.6, 7.5, 1.8, 1.0 Hz, 2H), 4.18 (qd, J=7.2, 1.1 Hz, 2H), 3.59-3.41 (m, 2H), 3.19 (ddq, J=38.7, 14.2, 7.1 Hz, 2H), 2.43 (dd, J=8.4, 6.2 Hz, 1H), 2.33 (s, 3H), 2.17 (dd, J=6.2, 4.9 Hz, 1H), 1.48 (dd, J=8.4, 4.9 Hz, 1H), 1.29 (t, J=7.1 Hz, 3H), 1.10 (t, J=7.1 Hz, 3H), 0.78 (t, J=7.1 Hz, 3H). ¹³C NMR (CDCl₃, 126 MHz): δ 170.8, 167.9, 139.1, 138.6, 128.8, 128.1, 127.3, 123.4, 61.1, 41.5, 39.4, 39.1, 28.3, 21.5, 21.3, 14.4, 13.2, 12.4. HRMS (m/z): calcd for C₁₈H₂₅O₃N, [M+H]⁺, 304.1913. found, 304.1917. GC: Using method described above, t_{R} (min): cis=9.80, trans=10.09. HPLC: Using method described above, t_{R} (min)=29.8. SFC: AS column, 2% IPA, 2.5 mL/min: λ=210 nm, t_{R} (min): major=7.66, minor=9.24.

Compound 8b.

¹H NMR (500 MHz, CDCl₃): δ 7.23-7.17 (m, 2H), 7.15-7.08 (m, 2H), 4.18 (qd, J=7.1, 1.6 Hz, 2H), 3.60-3.36 (m, 2H), 3.30-3.09 (m, 2H), 2.41 (dd, J=8.3, 6.2 Hz, 1H), 2.33 (s, 1H), 2.16 (dd, J=6.2, 4.9 Hz, 1H), 1.46 (dd, J=8.4, 4.8 Hz, 1H), 1.29 (t, J=7.1 Hz, 3H), 1.09 (t, J=7.1 Hz, 3H), 0.78 (t, J=7.1 Hz, 3H). ¹³C NMR (CDCl₃, 126 MHz): δ 170.9, 167.9, 137.0, 136.2, 129.6, 129.0, 128.8, 126.4, 61.0, 41.5, 39.4, 38.9, 28.3, 21.2, 21.1, 14.4, 13.2, 12.5. HRMS (m/z): calcd for C₁₈H₂₅O₃N, [M+H]⁺, 340.1913. found, 340.1917. GC: Using method described above, t_{R} (min): cis=9.93, trans=10.19. HPLC: Using method described above, t_{R} (min)=29.9. SFC: AS column, 2% IPA, 2.5 mL/min: λ=210 nm, t_{R} (min): major=8.85, minor=10.47.

Compound 8c.

¹H NMR (500 MHz, CDCl₃): δ 7.25 (d, J=8.9 Hz, 2H), 6.85 (d, J=8.9, 2H), 4.17 (qd, J=6.3, 5.5, 3.4 Hz, 2H), 3.80 (s, 3H), 3.54 (dq, J=15.0, 7.5 Hz, 1H), 3.45 (dq, J=14.1, 7.1 Hz, 1H), 3.20 (ddt, J=28.0, 14.2, 7.1 Hz, 2H), 2.37 (dd, J=8.2, 6.3 Hz, 1H), 2.14 (dd, J=6.3, 4.7 Hz, 1H), 1.45 (dd, J=8.2, 4.7 Hz, 1H), 1.29 (t, J=7.1 Hz, 3H), 1.09 (t, J=7.1 Hz, 3H), 0.79 (t, J=7.1 Hz, 3H). ¹³C NMR (CDCl₃, 126 MHz): δ 170.9, 168.0, 159.0, 131.3, 130.0, 127.9, 114.3, 61.0, 55.5, 41.5, 39.4, 38.6, 28.3, 21.0, 14.4, 13.3, 12.5. HRMS (m/z): calcd for C₁₈H₂₅O₄N, [M+H]⁺, 320.1862. found, 320.1866. GC: Using method described above, t_{R} (min): cis=10.56, trans=10.84. HPLC: Using method described above, t_{R} (min)=26.6. SFC: AS column, 4% IPA, 2.5 mL/min: λ=210 nm, t_{R} (min): major=6.49, minor=7.50.

Compound 8d.

¹H NMR (500 MHz, CDCl₃): δ 7.35-7.21 (m, 4H), 4.18 (qd, J=7.1, 1.6 Hz, 2H), 3.58-3.36 (m, 2H), 3.21 (ddq, J=37.3, 14.2, 7.1 Hz, 2H), 2.38 (dd, J=8.4, 6.2 Hz, 1H), 2.18 (dd, J=6.3, 5.0 Hz, 1H), 1.47 (dd, J=8.4, 5.0 Hz, 1H), 1.29 (t, J=7.1 Hz, 3H), 1.09 (t, J=7.1 Hz, 3H), 0.82 (t, J=7.1 Hz, 3H). ¹³C NMR (CDCl₃, 126 MHz): δ 170.5, 167.4, 137.8, 133.3, 130.2, 129.1, 128.6, 128.0, 61.2, 41.5, 39.5, 38.5, 28.5, 21.2, 14.4, 13.3, 12.4. HRMS (m/z): calcd for C₁₇H₂₂O₃C1N, [M+H]⁺, 324.1366. found, 324.1368. GC: Using method described above, t_{R} (min): cis=10.29, trans=10.56. HPLC: Using method described above, t_{R} (min)=31.1. SFC: AS column, 4% IPA, 2.5 mL/min: λ=210 nm, t_{R} (min): major=5.47, minor=5.91.

Compound 8e.

¹H NMR (500 MHz, CDCl₃): δ 7.62-7.56 (m, 2H), 7.47-7.36 (m, 2H), 4.20 (qd, J=7.1, 1.1 Hz, 2H), 3.60-3.42 (m, 2H), 3.22 (ddq, J=43.6, 14.2, 7.1 Hz, 2H), 2.45 (dd, J=8.4, 6.3 Hz, 1H), 2.24 (dd, J=6.3, 5.1 Hz, 1H), 1.54 (dd, J=8.5, 5.1 Hz, 1H), 1.30 (t, J=7.1 Hz, 3H), 1.11 (t, J=7.1 Hz, 3H), 0.82 (t, J=7.1 Hz, 3H). ¹³C NMR (CDCl₃, 126 MHz): δ 170.3, 167.1, 143.4, 129.8 (q, J=32.7 Hz), 126.9, 125.9 (q, J=3.7 Hz), 124.1 (q, J=271.9 Hz), 61.3, 41.5, 39.6, 38.7, 28.8, 21.4, 14.3, 13.3, 12.4. HRMS (m/z): calcd for C₁₈H₂₂F₃O₃N, [M+H]⁺, 358.1630. found, 358.1635. GC: Using method described above, t_{R} (min): cis=9.28, trans=9.54. HPLC: Using method described above, t_{R} (min)=31.8. SFC: AS column, 1% IPA, 2.5 mL/min: λ=210 nm, t_{R} (min): major=6.82, minor=7.41.

Compound 8f.

¹H NMR (500 MHz, CDCl₃): δ 7.85-7.78 (m, 3H), 7.74 (dt, J=1.4, 0.7 Hz, 1H), 7.53-7.43 (m, 3H), 4.22 (qd, J=7.2, 1.0 Hz, 2H), 3.65-3.43 (m, 2H), 3.23 (ddq, J=28.4, 14.1, 7.0 Hz, 2H), 2.57 (dd, J=8.4, 6.2 Hz, 1H), 2.26 (dd, J=6.2, 4.9 Hz, 1H), 1.60 (dd, J=8.4, 4.9 Hz, 1H), 1.32 (t, J=7.1 Hz, 3H), 1.12 (t, J=7.1 Hz, 3H), 0.74 (t, J=7.1 Hz, 3H). ¹³C NMR (CDCl₃, 126 MHz): δ 170.8, 167.7, 136.6, 133.5, 132.7, 128.8, 127.9, 127.7, 126.6, 126.2, 125.1, 124.8, 61.2, 41.5, 39.5, 39.3, 28.4, 21.3, 14.4, 13.3, 12.5. HRMS (m/z): calcd for C₂₁H₂₅O₃N, [M+H]⁺, 340.1913. found, 340.1917. GC: Using method described above, t_{R} (min): cis=11.63, trans=12.05. HPLC: Using method described above, t_{R} (min)=32.0. SFC: AS column, 7% IPA, 2.5 mL/min: λ=210 nm, t_{R} (min): major=6.02, minor=6.80.

Compound 10.

¹H NMR (500 MHz, CDCl₃): δ 7.33 (d, J=8.1 Hz, 2H), 7.13 (d, J=8.1 Hz, 2H), 4.19 (qd, J=7.1, 2.8 Hz, 2H), 4.15-4.02 (m, 2H), 2.33 (s, 3H), 2.20 (dd, J=8.5, 6.3 Hz, 1H), 2.08 (dd, J=6.3, 4.9 Hz, 1H), 1.48 (dd, J=8.5, 4.9 Hz, 1H), 1.29 (t, J=7.1 Hz, 3H), 1.19 (t, J=7.1 Hz, 3H). ¹³C NMR (CDCl₃, 126 MHz): δ 170.89, 169.9, 137.8, 135.6, 130.6, 129.9, 129.4, 129.2, 61.5, 61.1, 38.9, 28.1, 19.0, 14.39, 14.2. R_{f}=0.23 (silica gel, DCM). HRMS (m/z): calcd for C₁₆H₂₀O₄, [M+H]⁺, 277.1440. found, 277.1442. GC: Using method described above, t_{R} (min): cis=8.77, trans=9.00. SFC: AS column, 1% IPA, 2.5 mL/min: λ=210 nm, t_{R} (min): major=5.79, minor=7.63.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

INFORMAL SEQUENCE LISTING
CYP102A1
Cytochrome P450 (BM3)
Bacillus megaterium
GenBank Accession No. AAA87602
>gi|142798|gb|AAA87602.1| cytochrome P-450: NADPH-P-450 reductase precursor
[Bacillus megaterium]
SEQ ID NO: 1
TIKEMPQPK TFGELKNLPL LNTDKPVQAL MKIADELGEI FKFEAPGRVT RYLSSQRLIK
EACDESRFDK NLSQALKFVR DFAGDGLFTS WTHEKNWKKA HNILLPSFSQ QAMKGYHAMM
VDIAVQLVQK WERLNADEHI EVPEDMTRLT LDTIGLCGFN YRFNSFYRDQ PHPFITSMVR
ALDEAMNKLQ RANPDDPAYD ENKRQFQEDI KVMNDLVDKI IADRKASGEQ SDDLLTHMLN
GKDPETGEPL DDENIRYQII TFLIAGHETT SGLLSFALYF LVKNPHVLQK AAEEAARVLV
DPVPSYKQVK QLKYVGMVLN EALRLWPTAP AFSLYAKEDT VLGGEYPLEK GDELMVLIPQ
LHRDKTIWGD DVEEFRPERF ENPSAIPQHA FKPFGNGQRA CIGQQFALHE ATLVLGMMLK
HFDFEDHTNY ELDIKETLTL KPEGFVVKAK SKKIPLGGIP SPSTEQSAKK VRKKAENAHN
TPLLVLYGSN MGTAEGTARD LADIAMSKGF APQVATLDSH AGNLPREGAV LIVTASYNGH
PPDNAKQFVD WLDQASADEV KGVRYSVFGC GDKNWATTYQ KVPAFIDETL AAKGAENIAD
RGEADASDDF EGTYEEWREH MWSDVAAYFN LDIENSEDNK STLSLQFVDS AADMPLAKMH
GAFSTNVVAS KELQQPGSAR STRHLEIELP KEASYQEGDH LGVIPRNYEG IVNRVTARFG
LDASQQIRLE AEEEKLAHLP LAKTVSVEEL LQYVELQDPV TRTQLRAMAA KTVCPPHKVE
LEALLEKQAY KEQVLAKRLT MLELLEKYPA CEMKFSEFIA LLPSIRPRYY SISSSPRVDE
KQASITVSVV SGEAWSGYGE YKGIASNYLA ELQEGDTITC FISTPQSEFT LPKDPETPLI
MVGPGTGVAP FRGFVQARKQ LKEQGQSLGE AHLYFGCRSP HEDYLYQEEL ENAQSEGIIT
LHTAFSRMPN QPKTYVQHVM EQDGKKLIEL LDQGAHFYIC GDGSQMAPAV EATLMKSYAD
VHQVSEADAR LWLQQLEEKG RYAKDVWAG
CYP102A1
B. megaterium
>gi|281191140|gb|ADA57069.1| NADPH-cytochrome P450 reductase 102A1V9
[Bacillus megaterium]
SEQ ID NO: 2
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
CYP102A1
B. megaterium
>gi|281191138|gb|ADA57068.1| NADPH-cytochrome P450 reductase 102A1V10
[Bacillus megaterium]
SEQ ID NO: 3
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKEFVDWLDQASADEV
KGVRYSVFGCGDKNWATTYQKVPAFIDETFAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
CYP102A1
B. megaterium
>gi|281191126|gb|ADA57062.1| NADPH-cytochrome P450 reductase 102A1V4
[Bacillus megaterium]
SEQ ID NO: 4
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEATRVLVDPVPSYKQVKQLKYVGMVLNEALRLWPTAPAFSLYAKEDTVLGGEYPLEK
GDELMVLIPQLHRDKTIWGEDVEEFRPERFENPSAIPQHAFKPFGNGQRACIGQQFALHEATLVLGMMLK
HFDFEDHTNYELDIKETLTLKPEGFVVKAKSKKIPLGGIPSPSTEQSAKKVRKKVENAHNTPLLVLYGSN
MGTAEGTARDLADIAMSKGFAPQVATLDSHAGNLPREGAVLIVTASYNGHPPDNAKQFVDWLDQASADDV
KGVRYSVFGCGDKNWATTYQKVPAFIDETLAAKGAENIADRGEADASDDFEGTYEEWREHMWSDVAAYFN
LDIENSEDNKSTLSLQFVDSAADMPLAKMHGAFSANVVASKELQQPGSERSTRHLEIALPKEASYQEGDH
LGVIPRNYEGIVNRVTARFGLDASQQIRLEAEEEKLAHLPLGKTVSVEELLQYVELQDPVTRTQLRAMAA
KTVCPPHKVELEALLEKQAYKEQVLAKRLTMLELLEKYPACEMEFSEFIALLPSIRPRYYSISSSPRVDE
KQASITVSVVSGEAWSGYGEYKGIASNYLANLQEGDTITCFVSTPQSGFTLPKDSETPLIMVGPGTGVAP
FRSFVQARKQLKEQGQSLGEAHLYFGCRSPHEDYLYQEELENAQNEGIITLHTAFSRVPNQPKTYVQHVM
EQDGKKLIELLDQGAHFYICGDGSQMAPDVEATLMKSYADVYEVSEADARLWLQQLEEKGRYAKDVWAG
CYP102A1
B. megaterium
>gi|281191124|gb|ADA57061.1| NADPH-cytochrome P450 reductase 102A1V8
[Bacillus megaterium]
SEQ ID NO: 5
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPRVATLDSHAGNLPREGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
CYP102A1
B. megaterium
>gi|281191120|gb|ADA57059.1| NADPH-cytochrome P450 reductase 102A1V3
[Bacillus megaterium]
SEQ ID NO: 6
MTIKEMPQPKTFGELKNLPLLNTDKPVQALMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLVQKWERLNADEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKKIPLGGIPSPSTEQSAKKVRKKVENAHNTPLLVLYGSN
MGTAEGTARDLADIAMSKGFAPQVATLDSHAGNLPREGAVLIVTASYNGHPPDNAKQFVDWLDQASADDV
KGVRYSVFGCGDKNWATTYQKVPAFIDETLAAKGAENIADRGEADASDDFEGTYEEWREHMWSDVAAYFN
LDIENSEDNKSTLSLQFVDSAADMPLAKMHGAFSANVVASKELQQLGSERSTRHLEIALPKEASYQEGDH
LGVIPRNYEGIVNRVTARFGLDASQQIRLEAEEEKLAHLPLGKTVSVEELLQYVELQDPVTRTQLRAMAA
KTVCPPHKVELEALLEKQAYKEQVLAKRLTMLELLEKYPACEMEFSEFIALLPSISPRYYSISSSPHVDE
KQASITVSVVSGEAWSGYGEYKGIASNYLANLQEGDTITCFVSTPQSGFTLPKDSETPLIMVGPGTGVAP
FRGFVQARKQLKEQGQSLGEAHLYFGCRSPHEDYLYQEELENAQNEGIITLHTAFSRVPNQPKTYVQHVM
ERDGKKLIELLDQGAHFYICGDGSQMAPDVEATLMKSYADVYEVSEADARLWLQQLEEKGRYAKDVWAG
CYP102A1
B. megaterium
>gi|281191118|gb|ADA57058.1| NADPH-cytochrome P450 reductase 102A1V7
[Bacillus megaterium]
SEQ ID NO: 7
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPPEGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNEPKTYVQHVM
EQDGKKLIELLDQGAHFYICGDGSQMAPDVEATLMKSYAEVHQVSEADARLWLQQLEEKGRYAKDVWAG
CYP102A1
B. megaterium
>gi|281191112|gb|ADA57055.1| NADPH-cytochrome P450 reductase 102A1V2
[Bacillus megaterium]
SEQ ID NO: 8
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEATRVLVDPVPSYKQVKQLKYVGMVLNEALRLWPTAPAFSLYAKEDTVLGGEYPLEK
GDELMVLIPQLHRDKTIWGEDVEEFRPERFENPSAIPQHAFKPFGNGQRACIGQQFALHEATLVLGMMLK
HFDFEDHTNYELDIKETLTLKPEGFVVKAKSKKIPLGGIPSPSTEQSAKKVRKKVENAHNTPLLVLYGSN
MGTAEGTARDLADIAMSKGFAPQVATLDSHAGNLPREGAVLIVTASYNGHPPDNAKQFVDWLDQASADDV
KGVRYSVFGCGDKNWATTYQKVPAFIDETLAAKGAENIADRGEADASDDFEGTYEEWREHMWSDVAAYFN
LDIENSEDNKSTLSLQFVDSAADMPLAKMHGAFSANVVASKELQQLGSERSTRHLEIALPKEASYQEGDH
LGVIPRNYEGIVNRVTARFGLDASQQIRLEAEEEKLAHLPLGKTVSVEELLQYVELQDPVTRTQLRAMAA
KTVCPPHKVELEALLEKQAYKEQVLAKRLTMLELLEKYPACEMEFSEFIALLPSISPRYYSISSSPHVDE
KQASITVSVVSGEAWSGYGEYKGIASNYLANLQEGDTITCFVSTPQSGFTLPKDSETPLIMVGPGTGVAP
FRGFVQARKQLKEQGQSLGEAHLYFGCRSPHEDYLYQEELENAQNEGIITLHTAFSRVPNQPKTYVQHVM
ERDGKKLIELLDQGAHFYICGDGSQMAPDVEATLMKSYADVYEVSEADARLWLQQLEEKGRYAKDVWAG
CYP102A1
B. megaterium
>gi|269315992|gb|ACZ37122.1| cytochrome P450: NADPH P450 reductase
[Bacillus megaterium]
SEQ ID NO: 9
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
CYP102A1
B. megaterium
>gi|281191116|gb|ADA57057.1| NADPH-cytochrome P450 reductase 102A1V6
[Bacillus megaterium]
SEQ ID NO: 10
MTIKEMPQPKTFGELKNLPLLNTDKPVQALMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNADEHI
EVPEDMTRLTLDTIGLCGFNYRFNSFYRDQPHPFITSMVRALDEAMNKLQRANPDDPAYDENKRQFQDDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKQFVDWLDQASADEV
KGVRYSVFGCGDKNWATTYQKVPAFIDETLSAKGAENIAERGEADASDDFEGTYEEWREHMWSDLAAYFN
LNIENSEDNASTLSLQFVDSAADMPLAKMHGAFSANVVASKELQQPGSARSTRHLEIELPKEASYQEGDH
LGVIPRNYEGIVNRVTTRFGLDASQQIRLEAEEEKLAHLPLGKTVSVEELLQYVELQDPVTRTQLRAMAA
KTVCPPHKVELEALLEKQAYKEQVLTKRLTMLELLEKYPACEMEFSEFIALLPSMRPRYYSISSSPRVDE
KQASITVSVVSGEAWSGYGEYKGIASNYLAELQEGDTITCFVSTPQSGFTLPKDPETPLIMVGPGTGVAP
FRGFVQARKQLKEQGQSLGEAHLYFGCRSPHEDYLYQEELENAQNEGIITLHTAFSRVPNQPKTYVQHVV
EQDGKKLIELLDQGAHFYICGDGSQMAPDVEATLMKSYAEVHKVSEADARLWLQQLEEKSRYAKDVWAG
CYP102A1
B. megaterium
>gi|281191114|gb|ADA57056.1| NADPH-cytochrome P450 reductase 102A1V5
[Bacillus megaterium]
SEQ ID NO: 11
MTIKEMPQPKTFGELKNLPLLNTDKPVQALMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNADEHI
EVPEDMTRLTLDTIGLCGFNYRFNSFYRDQPHPFITSMVRALDEAMNKLQRANPDDPAYDENKRQFQDDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKQFVDWLDQASADEV
KGVRYSVFGCGDKNWATTYQKVPAFIDETLSAKGAENIAERGEADASDDFEGTYEEWREHMWSDLAAYFN
LNIENSEDNASTLSLQFVDSAADMPLAKMHGAFSANVVASKELQQPGSARSTRHLEIELPKEASYQEGDH
LGVIPRNYEGIVNRVTTRFGLDASQQIRLEAEEEKLAHLPLGKTVSVEELLQYVELQDPVTRTQLRAMAA
KTVCPPHKVELEALLEKQAYKEQVLTKRLTMLELLEKYPACEMEFSEFIALLPSMRPRYYSISSSPRVDE
KQASITVSVVSGEAWSGYGEYKGIASNYLAELQEGDTITCFVSTPQSGFTLPKDPETPLIMVGPGTGVAP
FRGFVQARKQLKEQGQSLGEAHLYFGCRSPHEDYLYQEELENAQNEGIITLHTAFSRVPNQPKTYVQHVV
EQDGKKLIELLDQGAHFYICGDGSQMAPDVEATLMKSYAEVHKVSEADARLWLQQLEEKSRYAKDVWAG

1. A reaction mixture for producing a cyclopropanation product comprising an olefinic substrate, a carbene precursor, and a cytochrome P450 BM3 enzyme variant, wherein the cytochrome P450 BM3 enzyme variant comprises a C400H mutation and one or more mutations selected from the group consisting of V78M, L181V, and L437M relative to the amino acid sequence set forth in SEQ ID NO:1.

2. The reaction mixture according to claim 1, wherein the cytochrome P450 BM3 enzyme variant comprises the C400H mutation and at least two mutations selected from the group consisting of V78M, L181V, and L437W.

3. The reaction mixture according to claim 1, wherein the cytochrome P450 BM3 enzyme variant comprises the C400H, V78M, L181V, and L437W mutations.

4. The reaction mixture according to claim 1, wherein the cytochrome P450 BM3 enzyme variant further comprises a T268A mutation.

5. The reaction mixture according to claim 1, wherein the olefinic substrate contains one or more electron withdrawing groups.

6. The reaction mixture according to claim 5, wherein the olefinic substrate is an acrylamide compound according to Formula I: 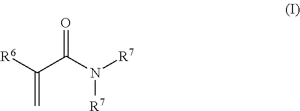
wherein:
each R⁷ is independently selected from the group consisting of H, optionally substituted C₁₋₁₈ alkyl, 2- to 18-membered heteroalkyl, hydroxyl, C₁₋₁₈ alkoxy, C₃₋₈ cycloalkyl, C₁₋₁₈ fluoroalkyl, optionally substituted C₆₋₁₀ aryl, optionally substituted 5- to 10-membered heteroaryl, or are taken together with the nitrogen atom to which they are bonded to form optionally substituted 5- to 10-membered heterocyclyl or optionally substituted 5- to 10-membered heteroaryl; and
R⁶ is selected from the group consisting of optionally substituted C₆₋₁₀ aryl and optionally substituted 5- to 10-membered heteroaryl.

7. The reaction mixture according to claim 6, wherein the olefinic substrate is selected from the group consisting of:

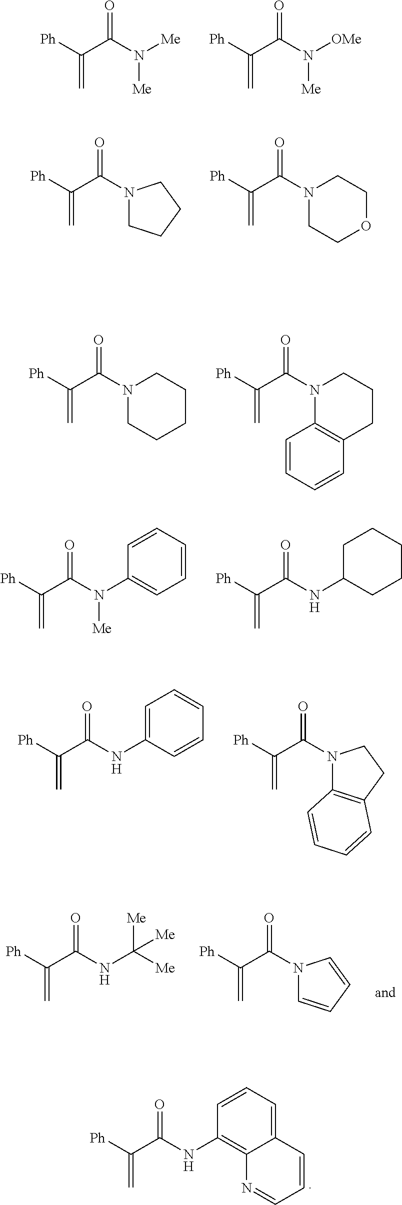

8. The reaction mixture according to claim 5, wherein the olefinic substrate is an acrylate compound according to Formula II: 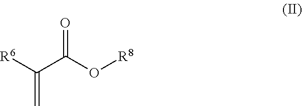
wherein
R⁸ is independently selected from the group consisting of H, optionally substituted C₁₋₁₈ alkyl, 2- to 18-membered heteroalkyl, C₃₋₈ cycloalkyl, C₁₋₁₈ fluoroalkyl, optionally substituted C₆₋₁₀ aryl, and optionally substituted 5- to 10-membered heteroaryl; and
R⁶ is selected from the group consisting of optionally substituted C₆₋₁₀ aryl and optionally substituted 5- to 10-membered heteroaryl.

9. The reaction mixture according to claim 8, wherein the olefinic substrate is selected from the group consisting of:

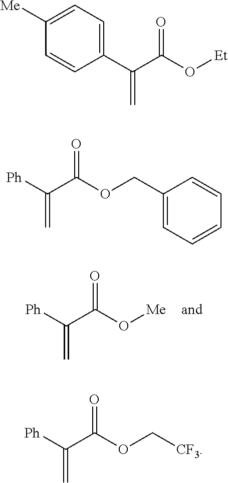 [SMILES: C=C(C(=O)OCC)c1ccc(C)cc1 ; C=C(C(=O)OCc1ccccc1)c1ccccc1 ; C=C(C(=O)OC)c1ccccc1 ; "and" ; C=C(C(=O)OCC(F)(F)F)c1ccccc1]

10. The reaction mixture according to claim 1, wherein the carbene precursor is a diazo reagent.

11. The reaction mixture according to claim 10, wherein the diazo reagent is selected from the group consisting of an α-diazoester, an α-diazoamide, an α-diazonitrile, an α-diazoketone, an α-diazoaldehyde, and an α-diazosilane.

12. The reaction mixture according to claim 11, wherein the diazo reagent is selected from the group consisting of: 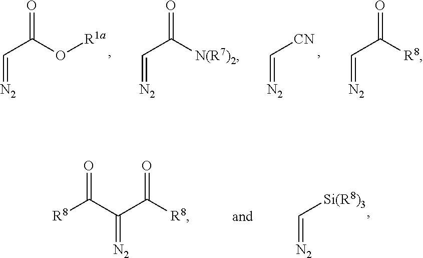
wherein
R^{1a} is selected from the group consisting of H and optionally substituted C₁₋₆ alkyl; and
each R⁷ and each R⁸ is independently selected from the group consisting of H, optionally substituted C₁₋₁₂ alkyl, optionally substituted C₂₋₁₂ alkenyl, and optionally substituted C₆₋₁₀ aryl.

13. The reaction mixture according to claim 11, wherein the diazo reagent is ethyl diazoacetate.

14. The reaction mixture according to claim 1, further comprising a reducing agent.

15. The reaction mixture according to claim 1, wherein the cytochrome P450 BM3 enzyme variant is localized within a whole cell and the cyclopropanation product is produced in vivo.

16. A method for producing a cyclopropanation product, the method comprising forming a reaction mixture comprising an olefinic substrate, a carbene precursor, and a cytochrome P450 BM3 enzyme variant under conditions sufficient to produce the cyclopropanation product, wherein the cytochrome P450 BM3 enzyme variant comprises a C400H mutation and one or more mutations selected from the group consisting of V78M, L181V, and L437M relative to the amino acid sequence set forth in SEQ ID NO:1.

17. The method according to claim 16, wherein the cytochrome P450 BM3 enzyme variant is localized within a whole cell and the cyclopropanation product is produced in vivo.

18. A cytochrome P450 BM3 enzyme variant comprising a C400H mutation and one or more mutations selected from the group consisting of V78M, L181V, and L437M relative to the amino acid sequence set forth in SEQ ID NO:1.

19. The cytochrome P450 BM3 enzyme variant according to claim 18, comprising the C400H mutation and at least two mutations selected from the group consisting of V78M, L181V, and L437W.

20. The cytochrome P450 BM3 enzyme variant according to claim 18, comprising the C400H, V78M, L181V, and L437W mutations.

21. The cytochrome P450 BM3 enzyme variant according to claim 18, further comprising a T268A mutation.


SEQUENCE LISTING (19 sequences)

SEQ ID NO: 1 - protein, 1048 aa, Bacillus megaterium
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 2 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 3 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKEFVDWLDQASADEVKGVRYSVFGCGDKNWATTYQKVPAFIDETFAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 4 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEATRVLVDPVPSYKQVKQLKYVGMVLNEALRLWPTAPAFSLYAKEDTVLGGEYPLEKGDELMVLIPQLHRDKTIWGEDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQPGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSIRPRYYSISSSPRVDEKQASITVSVVSGEAWSGYGEYKGIASNYLANLQEGDTITCFVSTPQSGFTLPKDSETPLIMVGPGTGVAPFRSFVQARKQLKEQGQSLGEAHLYFGCRSPHEDYLYQEELENAQNEGIITLHTAFSRVPNQPKTYVQHVMEQDGKKLIELLDQGAHFYICGDGSQMAPDVEATLMKSYADVYEVSEADARLWLQQLEEKGRYAKDVWAG

SEQ ID NO: 5 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPRVATLDSHAGNLPREGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 6 - protein, 1049 aa, Bacillus megaterium
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQLGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSISPRYYSISSSPHVDEKQASITVSVVSGEAWSGYGEYKGIASNYLANLQEGDTITCFVSTPQSGFTLPKDSETPLIMVGPGTGVAPFRGFVQARKQLKEQGQSLGEAHLYFGCRSPHEDYLYQEELENAQNEGIITLHTAFSRVPNQPKTYVQHVMERDGKKLIELLDQGAHFYICGDGSQMAPDVEATLMKSYADVYEVSEADARLWLQQLEEKGRYAKDVWAG

SEQ ID NO: 7 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPPEGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNEPKTYVQHVMEQDGKKLIELLDQGAHFYICGDGSQMAPDVEATLMKSYAEVHQVSEADARLWLQQLEEKGRYAKDVWAG

SEQ ID NO: 8 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEATRVLVDPVPSYKQVKQLKYVGMVLNEALRLWPTAPAFSLYAKEDTVLGGEYPLEKGDELMVLIPQLHRDKTIWGEDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQLGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSISPRYYSISSSPHVDEKQASITVSVVSGEAWSGYGEYKGIASNYLANLQEGDTITCFVSTPQSGFTLPKDSETPLIMVGPGTGVAPFRGFVQARKQLKEQGQSLGEAHLYFGCRSPHEDYLYQEELENAQNEGIITLHTAFSRVPNQPKTYVQHVMERDGKKLIELLDQGAHFYICGDGSQMAPDVEATLMKSYADVYEVSEADARLWLQQLEEKGRYAKDVWAG

SEQ ID NO: 9 - protein, 1049 aa, Bacillus megaterium; identical to SEQ ID NO: 2

SEQ ID NO: 10 - protein, 1049 aa, Bacillus megaterium
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNADEHIEVPEDMTRLTLDTIGLCGFNYRFNSFYRDQPHPFITSMVRALDEAMNKLQRANPDDPAYDENKRQFQDDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLSAKGAENIAERGEADASDDFEGTYEEWREHMWSDLAAYFNLNIENSEDNASTLSLQFVDSAADMPLAKMHGAFSANVVASKELQQPGSARSTRHLEIELPKEASYQEGDHLGVIPRNYEGIVNRVTTRFGLDASQQIRLEAEEEKLAHLPLGKTVSVEELLQYVELQDPVTRTQLRAMAAKTVCPPHKVELEALLEKQAYKEQVLTKRLTMLELLEKYPACEMEFSEFIALLPSMRPRYYSISSSPRVDEKQASITVSVVSGEAWSGYGEYKGIASNYLAELQEGDTITCFVSTPQSGFTLPKDPETPLIMVGPGTGVAPFRGFVQARKQLKEQGQSLGEAHLYFGCRSPHEDYLYQEELENAQNEGIITLHTAFSRVPNQPKTYVQHVVEQDGKKLIELLDQGAHFYICGDGSQMAPDVEATLMKSYAEVHKVSEADARLWLQQLEEKSRYAKDVWAG

SEQ ID NO: 11 - protein, 1049 aa, Bacillus megaterium; identical to SEQ ID NO: 10

SEQ ID NO: 12 - protein, 1048 aa, Artificial Sequence
  synthetic peptide construct - cytochrome P450 BM3 enzyme variant
TIKEMPQPKTFGELKNLPLLNTDKPVQALMKIADELGEIFKFEAPGRVTRYLSSQRLIKEACDESRFDKNLSQALKFXRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRAXDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAHIGQQFALHEATLVLGMMLKHFDFEDHTNYELDIKETXTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 13 - protein, 1048 aa, Artificial Sequence
  synthetic peptide construct - BM3-HStar
TIKEMPQPKTFGELKNLPLLNTDKPVQALMKIADELGEIFKFEAPGRVTRYLSSQRLIKEACDESRFDKNLSQALKFMRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRAVDEAMNKLQRANPDDPAYDENKRQFQEDIKVMNDLVDKIIADRKASGEQSDDLLTHMLNGKDPETGEPLDDENIRYQIITFLIAGHEATSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAHIGQQFALHEATLVLGMMLKHFDFEDHTNYELDIKETWTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 14 - protein, 1048 aa, Artificial Sequence
  synthetic peptide construct - cytochrome P450 BM3 enzyme variant C400, V78, L181, T268 and L437
TIKEMPQPKTFGELKNLPLLNTDKPVQALMKIADELGEIFKFEAPGRVTRYLSSQRLIKEACDESRFDKNLSQALKFXRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRAXDEAMNKLQRANPDDPAYDENKRQFQEDIKVMNDLVDKIIADRKASGEQSDDLLTHMLNGKDPETGEPLDDENIRYQIITFLIAGHEXTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAXIGQQFALHEATLVLGMMLKHFDFEDHTNYELDIKETXTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 15 - protein, 1048 aa, Artificial Sequence
  synthetic peptide construct - cytochrome P450 BM3 enzyme variant with substitutions F87V, P142S, T175I, A184V, S226R, H236Q, E252G, T268A, A290V, L353V, I366V, and/or E442K
TIKEMPQPKTFGELKNLPLLNTDKPVQALMKIADELGEIFKFEAPGRVTRYLSSQRLIKEACDESRFDKNLSQALKFVRDFAGDGLXTSWTHEKNWKKAHNILLPSFSQQAMKGYHAMMVDIAVQLVQKWERLNADEHIEVXEDMTRLTLDTIGLCGFNYRFNSFYRDQPHPFIXSMVRALDEXMNKLQRANPDDPAYDENKRQFQEDIKVMNDLVDKIIADRKAXGEQSDDLLTXMLNGKDPETGEPLDDXNIRYQIITFLIAGHEXTSGLLSFALYFLVKNPHVLQKXAEEAARVLVDPVPSYKQVKQLKYVGMVLNEALRLWPTAPAFSLYAKEDTVLGGEYPLEKGDEXMVLIPQLHRDKTXWGDDVEEFRPERFENPSAIPQHAFKPFGNGQRACIGQQFALHEATLVLGMMLKHFDFEDHTNYELDIKETLTLKPXGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 16 - protein, 1048 aa, Artificial Sequence
  synthetic peptide construct - cytochrome P450 BM3 enzyme variant with F87V, P142S, T175I, A184V, S226R, H236Q, E252G, T268A, A290V, L353V, I366V, and E442K substititions and optional V78M, L181V, C400H and/or L437M
TIKEMPQPKTFGELKNLPLLNTDKPVQALMKIADELGEIFKFEAPGRVTRYLSSQRLIKEACDESRFDKNLSQALKFXRDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAXDEVMNKLQRANPDDPAYDENKRQFQEDIKVMNDLVDKIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRAXIGQQFALHEATLVLGMMLKHFDFEDHTNYELDIKETXTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 17 - protein, 1048 aa, Artificial Sequence
  synthetic peptide construct - cytochrome P450 BM3 enzyme variant with V78, F87, P142, T175, L181, A184, S226, H236, E252, I263, T268, A290, A328, L353, I366, C400, L437, T438 and/or E442 substitutions
TIKEMPQPKTFGELKNLPLLNTDKPVQALMKIADELGEIFKFEAPGRVTRYLSSQRLIKEACDESRFDKNLSQALKFXRDFAGDGLXTSWTHEKNWKKAHNILLPSFSQQAMKGYHAMMVDIAVQLVQKWERLNADEHIEVXEDMTRLTLDTIGLCGFNYRFNSFYRDQPHPFIXSMVRAXDEXMNKLQRANPDDPAYDENKRQFQEDIKVMNDLVDKIIADRKAXGEQSDDLLTXMLNGKDPETGEPLDDXNIRYQIITFLXAGHEXTSGLLSFALYFLVKNPHVLQKXAEEAARVLVDPVPSYKQVKQLKYVGMVLNEALRLWPTXPAFSLYAKEDTVLGGEYPLEKGDEXMVLIPQLHRDKTXWGDDVEEFRPERFENPSAIPQHAFKPFGNGQRAXIGQQFALHEATLVLGMMLKHFDFEDHTNYELDIKETXXLKPXGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 18 - protein, 1048 aa, Artificial Sequence
  synthetic peptide construct - cytochrome P450 BM3 enzyme variant with L75, V78, F87, P142, T175, , M177, L181, A184, S226, H236, E252, I263, T268, A290, A328, L353, I366, C400, L437, T438 and/or E442 substitutions
TIKEMPQPKTFGELKNLPLLNTDKPVQALMKIADELGEIFKFEAPGRVTRYLSSQRLIKEACDESRFDKNLSQAXKFXRDFAGDGLXTSWTHEKNWKKAHNILLPSFSQQAMKGYHAMMVDIAVQLVQKWERLNADEHIEVXEDMTRLTLDTIGLCGFNYRFNSFYRDQPHPFIXSXVRAXDEXMNKLQRANPDDPAYDENKRQFQEDIKVMNDLVDKIIADRKAXGEQSDDLLTXMLNGKDPETGEPLDDXNIRYQIITFLXAGHEXTSGLLSFALYFLVKNPHVLQKXAEEAARVLVDPVPSYKQVKQLKYVGMVLNEALRLWPTXPAFSLYAKEDTVLGGEYPLEKGDEXMVLIPQLHRDKTXWGDDVEEFRPERFENPSAIPQHAFKPFGNGQRAXIGQQFALHEATLVLGMMLKHFDFEDHTNYELDIKETXXLKPXGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 19 - protein, 1048 aa, Artificial Sequence
  synthetic peptide construct - cytochrome P450 BM3 enzyme variant with all listed substitutions
TIKEMPQPKTFGELKNLPLLNTDKPVQALMKIADELGEIFKFEAPGXVTRYXSSQRLXKEACDESRFDKNLSQAXKFXRDXXGDGLXTSWTHEXNWKKAXNILLPXXSQQAMKGYHAMMVDIAVQLVQKWERLNXDEHIEVXEDMTRLTLDTIGLCGFNYRXNSFYRDQPHPFIXSXVRAXDEXMNKLQRANPDDPXYDENKRQXQEDIKVMNDLVDKIIADRKAXGEQSDDLLTXMLXGKDPETGEPLDDXNIXYQIITFLXAGHEXTSGLLXFALYFLVKNPHVLQKXAEEAARVLVDPVPSYKQVKQLKYVGMVLNEALRXWPTXPAFSLYAKEDTXLGGEYPLEKGDEXMVLIPQLHRDKTXWGDDVEEFRPERFENPSAIPQHAFKPFGNGQRAXIGQQFALHEATLVLGMMLKHFDFEDHTNYELDIXETXXLKPXGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG